(19) United States
(12) Patent Application Publication
(10) Pub. No.: US 2006/0134226 A1
(43) Pub. Date: Jun. 22, 2006

(54) Compositions useful to treat ocular neovascular diseases and macular degeneration

(76) Inventor: Todd Leonard, Minnetonka, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/280,960

(22) Filed: Nov. 16, 2005

Related U.S. Application Data
(60) Provisional application No. 60/628,162, filed on Nov. 16, 2004.

(51) Int. Cl. A61K 33/34; A61K 31/355; A61K 31/12; A61K 33/32

(52) U.S. Cl. 424/638; 424/641; 514/458; 514/474; 514/725; 514/690

(57) ABSTRACT

The present invention provides a composition that includes: (a) xanthophylls; (b) vitamin C; (c) vitamin E; (d) zinc; and (e) copper. The present invention also provides a method of treating macular degeneration in a human, inhibiting angiogenesis in a human, preventing impairment of the vision or for improving impaired vision of a human whose eye has drusen, and/or treating a disease associated with ocular neovascularitis in a human. The methods include administering to a human in need of such treatment an effective amount of the composition of the present invention.

### CROSS-REFERENCE TO RELATED APPLICATIONS

The present application claims priority to U.S. Provisional Application 60/628,162, filed on Nov. 16, 2004, which is incorporated herein by reference.

### BACKGROUND OF THE INVENTION

Macular degeneration associated with age and drusen is the leading cause of severe visual acuity loss in the United States and Western Europe in persons aged 55 years old or older. Age-related macular degeneration (AMD) is a collection of clinically recognizable ocular findings that can lead to blindness. The findings include drusen, retinal pigment epithelial (RPE) disturbance—including pigment clumping and/or dropout, RPE detachment, geographic atrophy, subretinal neovascularization and disciform scar. Not all these manifestations are needed for AMD to be considered present. The prevalence of persons with ophthalmoscopically or photographically identifiable drusen increases with age, and most definitions of AMD include drusen as a requisite. However, drusen alone do not seem to be directly associated with vision loss. It is rather, the association of drusen with the vision-threatening lesions of AMD, i.e., geographic atrophy, RPE detachment and subretinal neovascularization, that has led to their inclusion in the definition of AMD. Although recent studies have demonstrated the benefit of laser photocoagulation in those individuals with macular degeneration who develop acute, extrafoveal choroidal neovascularization, no treatment has been shown to be of benefit to the majority of people who have AMD. The cause of macular degeneration is unknown.

Recently, attention has been focused on the possible involvement of various minerals in retinal disease. Zinc has received particular notice in this regard due to the observation of high concentrations of zinc in ocular tissues, particularly the retina, pigment epithelium and choroid. Zinc is an important micronutrient that plays an essential role in human growth and function. Zinc is necessary for the activity of over a hundred enzymes, including carbonic anhydrase, superoxide dismutase and alkaline phosphatase. Zinc acts as a cofactor for numerous metalloenzymes, including retinol dehydrogenase and catalase. Zinc also is a cofactor in the synthesis of extracellular matrix molecules, is essential for cell membrane stability, is needed for normal immune function, is associated with melanin and is taken up in a facilitated manner by the retinal pigment epithelium. Despite the evidence supporting the notion that zinc must be essential to the metabolism of the retinochoroidal complex, relatively little is known of its role in the maintenance of normal eye function.

Toxicity from free radicals and oxidizers has also generated significant interest with regard to macular degeneration and the progression thereof. Circumstantial evidence indicates that protection against phototoxicity and oxidizers, such as would be provided by antioxidants, could slow the onset and progression of age-related macular degeneration as well as cataracts. If a treatment modality could slow down the progression of macular degeneration and/or cataracts, it would have a tremendous impact on the number of individuals who suffer from such problems due to the fact that such problems generally occur at significantly more advanced ages.

Accordingly, a need still exists in the art to provide methods and compositions for the treatment of macular degeneration and/or cataracts in the absence of surgery.

### SUMMARY OF THE INVENTION

The present invention provides a composition that includes: (a) xanthophylls; (b) vitamin C; (c) vitamin E; (d) zinc; and (e) copper. The composition can optionally include a pharmaceutically acceptable carrier, a neutraceutically acceptable carrier, alpha-lipoic acid, a phenolic compound, an anthocyanoside, beta-carotene, vitamin A, melanin, a melanin-promoting compound; a phytoestrogen selected from the group of genistein, genistin, 6″-O-Mal genistin, 6″-O—Ac genistin, daidzein, daidzin, 6″-O-Mal daidzin, 6″-O—Ac daidzin, glycitein, glycitin, 6″-O-Mal glycitin, biochanin A, formononetin; an antibiotic; an anti-inflammatory steroid; a non-steroidal anti-inflammatory drug (NSAID); a glutathione enhancing agent; an angiotensin converting enzyme inhibitor; selenium, manganese, glutathione (GSH), L-cysteine, pyridoxine (water soluble Vitamin B₆), riboflavin (water-soluble Vitamin B₂), bioflavenoid (Vitamin P); or any combination thereof.

The present invention also provides a method of treating macular degeneration (e.g., age-related macular degeneration (AMD), early onset macular degeneration, atrophic macular degeneration or neovascular macular degeneration) in a human. The present invention also provides a method of inhibiting angiogenesis in a human. The present invention also provides a method of preventing impairment of the vision or for improving impaired vision of a human whose eye has drusen. The present invention also provides a method of treating a disease associated with ocular neovascularitis in a human. The present invention also provides a method of treating cancer (e.g., colon, prostate, breast, cervical and skin), heart disease, and diabetes in a human. The present invention also provides a method of treating a vitamin deficiency in a human. The methods include administering to a human in need of such treatment an effective amount of the composition of the present invention.

### DETAILED DESCRIPTION OF THE INVENTION

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention relates to novel compositions, as well as novel methods of medical treatment. When describing the novel compositions and methods of medical treatment, the following terms have the following meanings, unless otherwise indicated.

As used herein, “lutein” refers to a compound having the formula: 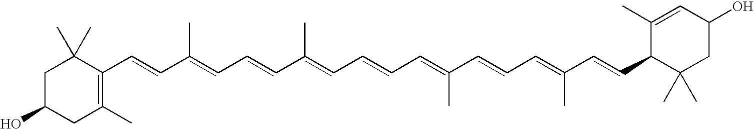
which is chemically designated as (3R,3′R,6′R)-β,ε-carotene-3,3′-diol. Lutein is a substance of a strongly marked yellow color, extracted from the yolk of eggs, and from the tissue of the corpus luteum. Lutein is not made in the body and must be obtained from food or vitamin supplements. Lutein is found in large amounts in green, leafy vegetables such as spinach; and legumes such as alfalfa.

The esterified form of lutein refers to a compound having the formula: 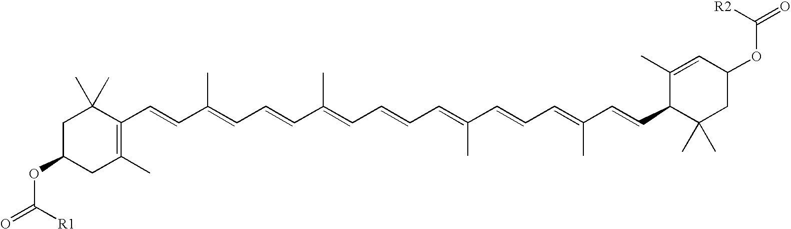
wherein R¹ and R² are radicals derived from fatty acids such as palmitic acid.

As used herein, “zeaxanthin” refers to a compound having the formula: 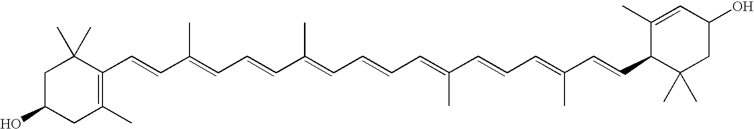
which is chemically designated as beta-carotene-3,3′-diol. Zeaxanthin is a carotene found in corn, fruits, seeds, and egg yolk.

As used herein, “xanthophylls” refers to any of several yellow accessory pigments which found in plant leaves, egg yolks and human blood plasma, these pigments are oxygenated derivatives of carotenes and are involved in photosynthesis, for example lutein, violaxanthin and neoxanthine. In one embodiment, the xanthophylls can be derived from alfalfa, clove, kale, spinach, squash, black bean tops, sea-weed, leafy green vegetable, or any combination thereof. In another specific embodiment, the xanthophylls can include, e.g., lutein and zeaxanthin. In another specific embodiment, the xanthophylls can include, e.g., free lutein and zeaxanthin.

As used herein, “fatty acids” refers to organic, monobasic acids derived from hydrocarbons by the equivalent of oxidation of a methyl group to an alcohol, aldehyde, and then acid. Fatty acids can be saturated, unsaturated, or partially unsaturated.

As used herein, “alfalfa” refers to lucern (Medicago sativa).

As used herein, “alpha carotene” refers to a compound of the formula:

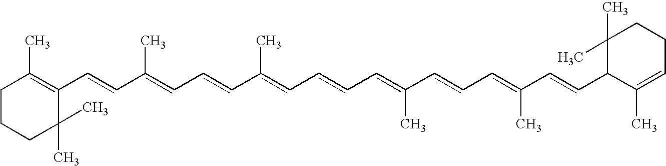

As used herein, “beta carotene” refers to a compound of the formula:

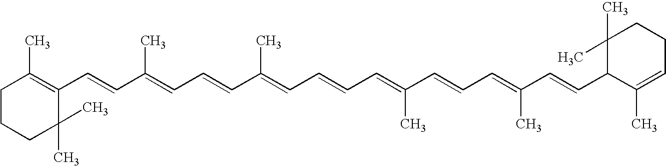

As used herein, “vitamin A” refers to a compound of the formula 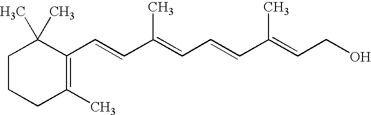
which is chemically designated as 3,7-dimethyl-9-(2,6,6-trimethyl-1-cyclohexen-1-yl)-2,4,6,8-nonatetraen-1-ol.

As used herein, “treating” or “treat” includes (i) preventing a pathologic condition (e.g., macular degeneration) from occurring (e.g. prophylaxis); (ii) inhibiting the pathologic condition (e.g., macular degeneration) or arresting its development; and (iii) relieving symptoms associated with the pathologic condition (e.g., macular degeneration).

As used herein, “cancer” includes a type of disease caused by cells that divide and grow uncontrollably, invading and disrupting other tissues and spreading to other areas of the body (metastasis). It is an abnormal uncontrolled growth of tissue that has potential to spread to distant sites of the body. Cancer exerts its deleterious effect on the body by: (a) Destroying the surrounding adjacent tissues: e.g. compressing nerves, eroding blood vessels, or causing perforation of organs; and (b) Replacing normal functioning cells in distant sites: e.g. replacing blood forming cells in the bone marrow, replacing bones leading to increased calcium levels in the blood, or in the heart muscles so that the heart fails.

As used herein, “ulcer” includes a sore, often deep, sometimes inflamed, which heals slowly or not at all.

As used herein, “macular degeneration” includes the breakdown or damage to a portion of the retina known as the macula. Symptoms include blurring of vision (in central visual field), colors appear dim and difficulty reading or performing work up close.

B Vitamins

The B vitamins are water-soluble. The B vitamins that can be present in the compositions of the present invention include thiamin (vitamin B1), riboflavin (vitamin B2), niacin (vitamin B3), pantothenic acid (vitamin B5), pyridoxine (vitamin B6), biotin, folic acid, the cobalamins (vitamin B12), and choline.

Vitamin B1 or thiamin helps keep collagen-rich connective and mucous membranes healthy, helps to maintain smooth muscles, helps in the formation of blood cells, and is necessary for proper nervous system function. When present in compositions of the instant invention, the vitamin B1 will be present in up to about 9 mg, in up to about 6 mg, or in up to about 3 mg.

Vitamin B2 or riboflavin is necessary for healthy hair, nails, and mucous membranes and is involved in red blood cell formation, antibody production, and overall growth. When present in compositions of the instant invention, the vitamin B2 will be present in up to about 30 mg, in up to about 20 mg, or in up to about 10 mg.

Vitamin B3 or niacin helps in the production of most of the sex hormones, dilates blood vessels, lowers cholesterol, and helps maintain blood circulation. Niacin is the generic name for a group of compounds which exhibit niacin activity, and includes niacinamide and nicotinic acid. When present in compositions of the instant invention, the vitamin B3 can be provided as niacinamide. When present in compositions of the instant invention, the vitamin B3 will be present in up to about 60 mg, in up to about 20 mg, or in up to about 10 mg.

Vitamin B6 or pyridoxine is involved in the production of ribonucleic acid (RNA) and deoxyribonucleic acid (DNA) and many other reactions in the body. Pyridoxine refers to and includes three different compounds: pyridoxine, pyridoxamine, and pyridoxal. When present in compositions of the instant invention, the vitamin B6 can be present in the form of pyridoxine hydrochloride. Additionally, when present in compositions of the instant invention, the vitamin B6 will be present in up to about 60 mg, in up to about 30 mg, or in up to about 20 mg.

Folic acid is essential in the production of red blood cells, the production of hormones, and the synthesis of DNA. When present in compositions of the instant invention, the folic acid will be present in up to about 2,400 mcg, in up to about 1,600 mcg, or in up to about 800 mcg.

Vitamin B12 or the cobalamins is necessary for overall metabolism, the function of the nervous system, metabolism of folic acid, and the production of red blood cells. There are at least three active forms of cobalamin: cyanocobalamin, hydroxocobalamin, and nitrocobalamin. When present in compositions of the instant invention, the vitamin B12 can be provided in the form of cyanocobalamin. Additionally, when present in compositions of the instant invention, the vitamin B12 can be present in up to about 1200 mcg, up to about 800 mcg, or up to about 400 mcg

Biotin is necessary for the metabolism of carbohydrates, proteins, and fats and is needed for healthy skin and hair. When present in compositions of the instant invention, the biotin can be present in the form of d-biotin. Additionally, when present in compositions of the instant invention, the biotin can be present in up to about 900 mcg, in up to about 600 mcg, or in up to about 300 mcg.

Pantothenic acid is important for the production of adrenal gland hormones, increases overall energy, and helps convert food into energy. When present in compositions of the instant invention, the pantothenic acid can be in the form of d-calcium pantothenate. Additionally, when present in compositions of the instant invention, the pantothenic acid can be present in up to about 30 mg, in up to about 20 mg, or in up to about 10 mg.

Choline is necessary for nervous system function and brain function. It is also important for gall bladder and liver function. When present in compositions of the instant invention, the choline can be provided in the form choline bitartrate. Additionally, when present in compositions of the instant invention, the choline can be present in up to about 1500 mg, in up to about 1000 mg, or in up to about 500 mg.

Vitamin C

Vitamin C is a well known water-soluble antioxidant. Humans depend on external sources of vitamin C to meet their vitamin C requirements. Vitamin C in the form of ascorbate is found in the aqueous humor of human eyes. A high concentration of ascorbate in the aqueous humor of eyes is maintained by active transport of ascorbate from the blood stream to the posterior chamber of the eyes. Maximum aqueous humor ascorbate concentration occurs with a blood plasma ascorbate level in the range of approximately 0.3 to 0.5 milligrams/deciliter (mg/dl).

The U.S. Recommended Daily Allowance (RDA) for vitamin C in the form of ascorbic acid is 60 mg. Very large daily doses of vitamin C have been taken over many years with no or only minor undesirable effects. Intakes of 1,000 mg or more of vitamin C can be consumed daily without any known adverse effects.

Ascorbic acid is the preferred source of vitamin C in the compositions of the present invention, although other sources such as for example sodium ascorbate could alternatively be used.

Vitamin D

Vitamin D is an essential vitamin. Vitamin D assists in the mineralization and calcification of bone, prevents rickets in children, prevents osteomalacia in adults, preserves bone and tooth growth, and lowers blood pressure. Vitamin D is fat soluble. When present in compositions of the instant invention, the Vitamin D will be present in up to about 800 I.U., in up to about 600 I.U., or in up to about 400 I.U.

Vitamin E

Vitamin E is also a well-known antioxidant. Vitamin E can work synergistically with vitamin C in protecting vital cell function from normal oxidants. Vitamin E is a relatively non-toxic fat-soluble vitamin. Vitamin E is readily oxidized thereby significantly reducing its activity during periods of storage prior to ingestion. Once ingested, vitamin E is stored within the body and can contribute to the total body pool of vitamin E for up to one year.

The Recommended Daily Allowance (RDA) of vitamin E in the form of dl-alpha tocopheryl acetate is 30 IU. No adverse effects of dl-alpha tocopheryl acetate have been observed at levels as high as 800 mg, with 1.0 mg of dl-alpha tocopheryl acetate being equal to 1 IU of dl-alpha tocopheryl acetate.

D1-alpha tocopheryl acetate is the preferred source of vitamin E in the subject tablets although other sources of vitamin E, such as for example trimethyl tocopheryl acetate and/or vitamin E succinate, may be used in the alternative.

Vitamin K

Vitamin K is an active blood clotting agent and assists in bone formation. When present, the compositions of the instant invention can include up to about 75 mcg of Vitamin K, up to about 50 mcg of Vitamin K, or up to about 25 mcg of vitamin K.

Beta-Carotene

Beta-carotene, a proform of vitamin A, is a lipid-soluble orange pigment found in many vegetables. Beta-carotene is converted to vitamin A in the body with an efficiency of approximately 50 percent. The Recommended Daily Allowance (RDA) of vitamin A is 5,000 IU. Beta-carotene has one of the highest antioxidant potentials of the antioxidants. No observed adverse effects are observed for beta-carotene at dosage levels as high as 25 mg per day for healthy, non-smokers. However, an increased risk of fatal coronary heart attacks in men with previous myocardial infarction and an increased risk of lung cancer among male smokers has been observed in individuals who receive 20 mg/day of beta-carotene.

Beta-carotene is preferred in the subject composition due to its ready commercial availability although alternative carotenoid proforms of vitamin A could likewise be used.

Zinc

Zinc is important in maintaining the health of an eye's retina and is an essential part of more than 100 enzymes involved in digestion, metabolism, reproduction and wound healing. The Recommended Daily Allowance (RDA) for zinc is approximately 15 mg. In one study, 80 mg of zinc was shown to be significantly better than placebo in retarding macular degeneration changes. (Newsome, Arch Ophthalmol 106:192-8, 1988.) About 200 mg dosage of zinc per day, although well tolerated, has been shown to have potential side effects such as anemia. The anemia associated with high dosage zinc intake is attributable to copper deficiency. Diet supplementation with copper does not appear to have a deleterious effect on zinc absorption.

Zinc is preferred in the form of zinc oxide in subject tablets due to the fact zinc oxide provides the most concentrated form for elemental zinc and is well tolerated in the digestive system. However, other forms of zinc such as for example zinc gluconate may alternatively be used or be used in combination with zinc oxide in the subject composition.

Copper

Copper, like zinc, is another important cofactor for metalloenzymes, and is a second necessary cofactor for superoxide dismutase. Two mg is the Recommended Daily Allowance (RDA) for copper.

Copper in the form of cupric oxide is preferred in the subject tablets to help prevent zinc induced copper deficiency anemia, although other forms of copper such as for example copper gluconate may alternatively be used or used in combination with cupric oxide in the subject composition.

Lutein

Lutein, like beta-carotene, is a carotenoid. Lutein is one of the most abundant carotenoids found in fruits and vegetables. Lutein is also an antioxidant found in the retina of healthy eyes. As with beta-carotene, lutein is subject to degradation during periods of storage prior to ingestion. Accordingly, quantities larger than the desired daily dosage quantity of lutein can typically be used in a composition of the invention. For example, at least about 50 mcg, at least about 100 mcg, at least about 250 mcg, at least about 500 mcg or at least about 1,000 mcg of lutein can be present in the compositions of the present invention.

Zeaxanthine

Zeaxanthine, like lutein and beta-carotene, is a carotenoid. Zeaxanthine is found naturally in fruits and vegetables. Zeaxanthine is also an antioxidant found in the retina of healthy eyes. As with beta-carotene, zeaxanthine is subject to degradation during periods of storage prior to ingestion. Accordingly, quantities larger than the desired daily dosage quantity of zeaxanthine can typically be used in a composition of the invention. For example, at least about 50 mcg, at least about 100 mcg, at least about 250 mcg, at least about 500 mcg or at least about 1,000 mcg of zeaxanthine can be present in the compositions of the present invention.

Lutein-Zeaxanthine

Lutein-zeaxanthine raw material combinations achieved deliberately, because of normal composition, or through raw material contamination may likewise be included in the compositions of the present invention. Specific ratios of lutein-zeaxanthine include, e.g., about 99:1 wt. % lutein to zeaxanthine; to about 99:1 wt. % zeaxanthine to lutein.

Alpha-Lipoic Acid

Alpha-lipoic acid provides superior antioxidant protection due to the fact that it enhances the potency of other antioxidants in the body. Alpha-lipoic acid may be added to the nutritional or dietary supplement composition of the present invention if desired. Suitable compositions of the present invention can include, e.g., about 0.25 to about 5 mg of alpha-lipoic acid, for a total daily dosage of about 1 to about 20 mg.

Phenolic Compounds

Phenolic compounds such oligomeric proanthocyanidins are additional useful antioxidants. Oligomeric proanthocyanidins are found naturally in grape seeds. Phenolic compounds may be added to the nutritional or dietary supplement composition of the present invention if desired. Suitable compositions of the present invention can include, e.g., about 0.25 to about 5 mg of phenolic compounds, for a total daily dosage of about 1 to about 20 mg.

Anthocyanosides

Anthocyanosides are useful antioxidants found naturally in bilberry fruit. Anthocyanosides may be added to the nutritional or dietary supplement composition of the present invention if desired. Suitable compositions of the present invention can include, e.g., about 0.25 to about 5 mg of anthocyanosides, for a total daily dosage of about 1 to about 20 mg.

Melanin

It will be appreciated that the term “melanin” as used herein refers to both soluble and insoluble forms of melanin, including eumelanin and phaeomelanin, and precursors or fragments of these molecules. The term “melanin-promoting compound” as used herein refers to any compound capable of increasing the amount or activity of melanin in vivo. Examples of melanin-promoting compounds are tyrosinase, melanocyte stimulating hormone (MSH), melanocyte concentrating hormone (MCH), minocycline, latanoprost, melanotan-I, prostaglandins and compounds with prostaglandin activity, ACTH, melanocortin receptor antagonists, endothelin, rifabutin, diacycloglycerols, arbutin, amiodarone, pefloxcin, chlorpromazine, desipramine, sulfasalazine, zidovudine, clofazimine, bergapten, metenkephalin and cyclophosphamide. Such alternative compounds may modify the production or bioactivity of melanin.

Lycopene

Lycopene has been found to reduce the risk of cancer and has antioxidant capabilities. Lycopene is found primarily in tomatoes, red grapefruit, watermelon, and other sources, and is a carotenoid. When present in compositions of the instant invention, the lycopene can be obtained from tomatoes. Additionally, when present in compositions of the instant invention, the lycopene can be present in up to about 20 mg, in up to about 10 mg, or in up to about 5 mg.

Co-Enzyme Q10

Co-enzyme Q10, also known as ubiquinone, is an antioxidant which protects the body from radicals. Co-enzyme Q10 aids metabolic reactions, such as the complex process of transforming food into ATP, and helps people with congestive heart failure and angina. Co-enzyme Q10 is depleted in people taking lovastatin and pravastatin, which are cholesterol lowering drugs. When present in compositions of the instant invention, the of Co-enzyme Q10 can be present in up to about 300 mg, in up to about 200 mg, or in up to about 100 mg.

Phytoestrogens

A select group of phytoestrogens—genistein, genistin, 6″-O-Mal genistin, 6″-O—Ac genistin, daidzein, daidzin, 6″-O-Mal daidzin, 6″-O—Ac daidzin, glycitein, glycitin, 6″-O-Mal glycitin, biochanin A, and formononetin, shown in Formulas 1 and 2 below—are effective in inhibiting or reducing the risk of development of macular degeneration.

The phytoestrogens of Formulas 1 and 2 exhibit estrogenic activity, a protective factor against macular degeneration. The estrogenic activity of the phytoestrogens is effective to lower serum LDL cholesterol levels in the blood. The reduction of LDL cholesterol levels in the blood reduces the amount of LDL cholesterol available for oxidation in the macula, thereby reducing the accumulation of oxidized lipid products in the retinal pigment epithelium. The reduction of LDL cholesterol levels in the blood also reduces atherosclerotic plaque buildup in the retinal and subretinal blood vessels, reducing hypoxia in retinal and subretinal tissues, and ultimately inhibiting the growth of new blood vessels.

Advantageously, specific phytoestrogens in the group have further protective functionality against macular degeneration. Some, such as genistein and daidzein, are effective antioxidants. The antioxidant activity of these phytoestrogens is effective to reduce the amounts of reactive oxidized species in the macula, thereby inhibiting the damage that reactive oxidized species cause to the retinal and subretinal tissues. Some, particularly genistein, are tyrosine kinase inhibitors which inhibit angiogenesis in the retinal pigment epithelium and the choroid. The tyrosine kinase inhibiting activity of these phytoestrogens interferes with the activation of existing endothelial cells by cytokine growth factors, particularly bFGF, thereby preventing the development of new blood vessels by activated endothelial cells. Some, including genistein and daidzein, have vasodilating activity which induces the dilation of retinal and subretinal vasculature, permitting more blood flow through the vessels and reducing the incidence of hypoxia. Other phytoestrogens of Formulas 1 and 2 are metabolized in humans to phytoestrogens having antioxidant activity, tyrosine kinase inhibiting activity, or vasodilation activity, for example biochanin A is metabolized to genistein and formononetin is metabolized to daidzein.

To inhibit or reduce the risk of macular degeneration, a composition containing at least one phytoestrogen selected from the phytoestrogen isoflavone compounds of Formulas 1 and 2 is administered to a human, in an amount effective to elevate the level of the phytoestrogen(s) in the blood. An elevated level of phytoestrogen(s) in the blood is indicated by a blood concentration of a combination of the phytoestrogen(s) and its/their metabolites of at least 50 ng/ml, and more preferably a blood concentration of at least 50 ng/ml of the phytoestrogen(s) itself/themselves. Primary metabolites of the phytoestrogens of Formulas 1 and 2 in humans are equol, angolensin, O-desmethylangolensin, dihydrodaidzein, dehydroequol, 2-dehydro-O-desmethylangolensin, tetrahydrodaidzein, dihydrogenistein, and 6″-hydroxy-O-desmethylangolensin. Preferably the composition containing the phytoestrogen(s) is a pharmaceutical composition or a dietary composition.

| | |
|---|---|
| | Formula 1 |
| 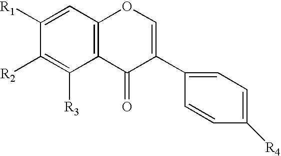 | |

| Compound | R₁ | R₂ | R₃ | R₄ |
|---|---|---|---|---|
| Genistein | OH | H | OH | OH |
| Daidzein | OH | H | H | OH |
| Glycitein | OH | OCH₃ | H | OH |
| Biochanin A | OH | H | OH | OCH₃ |
| Formononetin | OH | H | H | OCH₃ |

| | |
|---|---|
| | Formula 2 |
| 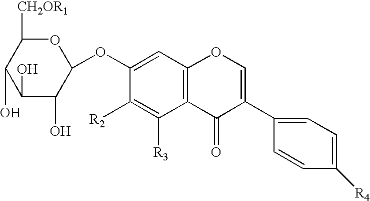 | |

| Compound | R₁ | R₂ | R₃ | R₄ |
|---|---|---|---|---|
| Genistin | H | H | OH | OH |
| 6′-OMal genistin | COCH₂CO₂H | H | OH | OH |
| 6′-OAc genistin | COCH₂ | H | OH | OH |
| Daidzin | H | H | H | OH |
| 6′-OMal daidzin | COCH₂CO₂H | H | H | OH |
| 6′-OAc daidzin | COCH₃ | H | H | OH |
| Glycitin | H | OCH₃ | H | OH |
| 6′-OMal glycitin | COCH₃ | OCH₃ | H | OH |

Angiotensin Converting Enzyme Inhibitor

The angiotensin converting enzyme inhibitor useful in the compositions and methods of this invention is selected from: enalapril, enalaprilat, lisinopril, captopril, ranipril, perindopril, zofenopril, quinapril, pentopril, cilazapril, pivopril, fosenopril, indolapril, indalapril, phenacein, fentiapril, alacepril, perinodopril, mugenic acid, ancovenin; 2-[2-[[1-(1-(ethoxycarbonyl)-3-phenylpropyl]amino]-1-oxopropyl-6,7-dimethoxy-1,2,3,4-tetrahydro-3-isoquinolinecarboxylic acid; 3-[[1-ethoxycarbonyl-3-phenyl-(1S)propyl]amino]-2,3,4,5-tetrahydro-2-oxo-1-(3S)benzazepine-1-acetic acid HCl; 3-[[1-carboxylate-3-phenyl-1(S)propyl]-amino]-2,3,4,5-tetrahydro-2-oxo-1(3S)benzazepine-1-acetic acid HCl; 2[2-[[1-carbonyl-3-phenylpropyl]-amino]-1-oxopropyl]-1,2,3,4-tetra-hydro-3-isoquinoline-carboxylic acid; [1-(S)4S]4-[[2-[6-(aminosulfonyl)-7-chloro-1,2,3,4-tetrahydro-4-oxo-2-quinazolinyl]ethyl]-thio]-1-(3-mercapto-2-methyl-1-oxo-propyl)-L-proline monosodium salt; [1-(s)4S]4-[4-[6-(aminosulfonyl)-7-chloro-1,2,3,4-tetrahydro-4-oxo-2-quinazolinylphenoxy]-1-(3-mercapto-2-methyl-1-oxopropyl)-L-proline; calcium N[(S)3-(N-cyclohexane-carbonyl-D-alanylthio)-2-methylpropanoyl]-L-prolinate; (−)-(S)1-[(S)3-mercapto-2-methyl-1-oxopropyl]indoline-2-carboxylic acid; and 3-[(5-amino-1-carboxy-1Spentyl)-amino]-2,3,4,5-tetrahydro-2-oxo-3S1H1-benzazepine-1-acetic acid.

An effective amount of angiotensin converting enzyme inhibitor can be about 0.1 mg to about 1 g, and preferably about 5 to 500 mg per day. The dosage regimen can be one to four times a day depending on the daily total required and the unit dosage.

Selenium

Selenium is a metal which has been known to be markedly deficient in cataracts versus clear lenses Swanson, Biochem Biophy Res Comm 45:1488-96, 1971. Selenium is a necessary cofactor for metalloenzymes, particularly GSHPx, which scavenges peroxides. Chaney, at p. 988. A past study showed macular degeneration being inversely related with plasma activity of GSHPx and suggested that its activity is an indication of the adequacy of selenium nutritional status. Weiter, Invest Ophthalmol (Supp) 26:58, 1985. Other studies have documented that selenium deficiency results in markedly decreased activity of lens GSHPx in animals, and the addition of selenium in selenium deficient animals blocked cataract formation. Whanger, Nutr Rep Int 12:343, 1975; Lawrence, Exp Eye Res 18:563, 1974.

The presently-accepted safe and adequate daily dietary intake of selenium is about 50 to 200 micrograms (mcg) for an adult. There is no U.S. Recommended Daily Allowance (RDA) for selenium. Typical dietary intake for adults is in the lower end of the above range. A presently accepted, estimated maximum safe daily selenium human intake is 5 micrograms per kilogram of body weight per day. In the present composition, selenium can added at about 12.5 mcg/g, or a total of 100 mcg/day.

Manganese

In general, manganese concentration has been known to decrease in cataracts versus clear lenses, although not nearly as dramatically as copper, zinc and selenium. Swanson, Biochem Biophy Res Comm 45:1488-96, 1971. Manganese is an important cofactor for metalloenzymes Orten, at pp. 725-6. As briefly noted above, a second type of superoxide dismutase exists in the mitochondria and has manganese as a necessary cofactor. Another metalloenzyme, to which manganese is a cofactor, is methionine adenosyltransferase, which is found in the lens. See generally Geller, Exp. Eye. Res. 43:998, 1986.

There is no presently known minimum daily requirement of manganese. However, a daily dose of 10 mg is an accepted safe amount and commonly available in the supermarket. Preferably, manganese is provided in the present composition at about 1.25 mg/g, or a total of 10 mg/day.

L-Cysteine

Glutathione (GSH), a tripeptide which includes L-cysteine, has been called the Achilles' heel of the human lens system. Cole, JAMA 254:1008, 1985. It, as alluded to above, acts directly as an antioxidant intracellularly and is also an important constituent of many enzymes, not the least of which is GSHPx, which reduces the potent oxidizer—peroxide. Reddy, Exp Eye Res 11:310-28, 1971; Bhuyan, Biochem Biophys Acta 497:641-51, 1977; Kinoshita, Am J Ophthalmol 46:36-41, 1958; Pirie, Biochem J 96:244-53, 1965. Glutathione has been known to decrease in concentration in human cataracts. Consul, Eye, Ear, Nose and Throat Monthly 47:77-80, 1968. Of the three constituent amino acids, L-cysteine is the one which has been thought to be rate limiting in regard to glutathione synthesis. Rathbun, In: Hockwin 0 (Ed.) Altern der Linse, Wilhelm Mayr, 1982, pp. 169-74.

L-cysteine is a naturally occurring amino acid. A total dose of 400 mg per day of L-cysteine is readily available to someone on a high protein diet. The present composition uses L-cysteine at about 50 mg/g, or a total of about 400 mg/day.

Pyridoxine

Pyridoxine, or water soluble Vitamin B₆, is known to be important for protein synthesis in general and may enhance glutathione production. Chaney, at pp. 976-8. The U.S. Recommended Daily Allowance (RDA) for Vitamin B₆ is 2 mg/day. Due to the known importance of glutathione in maintaining lens clarity, pyridoxine is added to the present composition in the dose of about 6.25 mg/g, or 50 mg/day. Although the dose is much greater than the minimum daily requirement, it is apparently safe and is not an uncommon dose in multivitamins available in drugstores or grocery stores.

Riboflavin

Riboflavin, or water-soluble Vitamin B₂, has previously shown a good correlation with riboflavin nutritional status in older patients between those who had clear lenses and those who had cataracts. Skalka, Metabolic Ped Ophthalmol 5:17-20, 1981; Bhat, Nutr Rep Int 36:685, 1987. Glutathione reductase is necessary to reduce glutathione after oxidation, and riboflavin deficiency is associated with decreased glutathione reductase activity. Srivastava, Exp Eye Res 16:519, 1973. This enzyme is lower in cataractous lenses and would appear to be necessary if the glutathione system is to operate as an antioxidant. Beutler, Science 165:613-5, 1969; Day, Am J Ophthalmol 14:1005-9, 1931; Ono, Internat J Vit Nutr Res 46:422-6, 1976; Yagi 10th International Congress of Nutrition, Abstract No. 32-11, p. 169 (August 1975). 40 mg a day is a common dosage of riboflavin and is available in supermarkets. The U.S. Recommended Daily Allowance (RDA) is about 1.7 mg. Preferably, about 7.5 mg/g is used in the present composition, or a total of 60 mg/day.

The present composition may also include bioflavenoid (Vitamin P).

Antibiotic

As used herein, an “antibiotic” is any compound having activity against either Gram-positive or Gram-negative organisms (i.e., inhibits the growth or destroys the development of either Gram-positive or Gram-negative organisms). Stedman's Medical Dictionar, Illustrated, (25th Ed.), Williams & Wilkins: Baltimore (1990) and Mosby's Medical, Nursing, & Allied Health Dictionary, (5th Ed.), Mosby: St. Louis (1998).

Suitable antibiotics are disclosed, e.g., in Physician's Desk Reference (PDR), Medical Economics Company (Montvale, N.J.), (53rd Ed.), 1999; Mayo Medical Center Formulary, Unabridged Version, Mayo Clinic (Rochester, Minn.), January 1998; Merck Index, An Encyclopedia of Chemicals, Drugs, and Biologicals, (11th Ed.), Merck & Co., Inc. (Rahway, N.J.), 1989; University of Wisconsin Antimicrobial Use Guide, http://www.medsch.wisc.edu/clinsci/amcg/amcg.html; Introduction on the Use of the Antibiotics Guideline, Descriptions of Specific Antibiotic Classes, Thomas Jefferson University, http://jeffline.tju.edu/CWIS/OAC/antibiotics_guide/intro.html; and references cited therein.

Suitable antibiotics include, e.g., aminoglycosides, β-lactam antibiotics, cephalosporins, macrolides, miscellaneous antibiotics, penicillins, tetracyclines, antifungals, antimalarial agents, antituberculosis agents, antivirals, leprostatics, miscellaneous anti-infectives, quinolones, sulfonamides, urinary anti-infectives, nasal antibiotics, opthalmic antibiotics, opthalmic antivirals, opthalmic quinalones, opthalmic sulfonamides, skin and mucous membrane antibiotics, skin and mucous membrane antifungals, skin and mucous membrane antivirals, skin and mucous membrane miscellaneous anti-infectives, skin and mucous membrane scabicides and pedulicides, skin and mucous membrane antinepolasts, nitrofurans, and oxazolidinones. Physician's Desk Refernce (PDR), Medical Economics Company (Montvale, N.J.), (53rd Ed.), 1999 and Mayo Medical Center Formulary, Unabridged Version, Mayo Clinic (Rochester, Minn.), January 1998.

Aminoglycosides include, e.g., Amikacin (amikacin sulfate); Garamycin (gentamicin sulfate); Nebcin (tobramycin sulfate); Netromycin (netilmicin sulfate); Streptomycin Sulfate; and TOBI (tobramycin).

β-Lactam antibiotics include, e.g., Azactam (aztreonam); Cefotan (cefotetan); Lorabid (loracarbef); Mefoxin (cefoxitin); Merrem (meropenem); and Primaxin (imipenem and cilastatin for injectable suspension).

Cephalosporins include, e.g., Ancef (cefazolin); Ceclor (cefaclor); Cedax (ceftibuten); Cefizox (ceftizoxime sodium); Cefobid (cefoperazone sodium); Ceftin (cefuroxime axetil); Cefzil (cefprozil); Ceptaz (ceftazidime); Claforan (cefotaxime); Duricef (cefadroxil monohydrate); Fortaz (ceftazidime); Keflex (cephalexin); Keftab (cephalexin HCl); Kefuirox (cefuiroxime); Kefzol (cefazolin); Mandol (cefamandole nafate); Maxipime (cefepime HCl); Monocid (cefonicid sodium); Omnicef (cefdinir); Rocephin (ceftriaxone); Suprax (cefixime); Tazicef (ceftazidime); Tazidime (ceftazidime); Vantin (cefpodoxime proxetil); and Zinacef (cefuiroxime).

Macrolides include, e.g., Biaxin (clarithromycin); Dynabac (dirithromycin); E.E.S. 200 (Erythromycin Ethylsuccinate); E.E.S. 400 (Erythromycin Ethylsuccinate); Ery-Ped 200 (Erythromycin Ethylsuccinate); EryPed 400 (Erythromycin Ethylsuccinate); Ery-Tab (Erythromycin delayed-release tablets); Erythrocin Stearate (Erythromycin stearate); Ilosone (erythromycin estolate); PCE Dispertab (erythromycin particles in tablets); Pediazole (erythromycin ethylsuccinate and sulfisoxazole acetyl for oral suspension); Tao (troleandomycin); Zithromax (azithromycin); and Erythromycin.

Miscellaneous antibiotics include, e.g., Cleocin HCl (clindamycin hydrochloride); Cleocin Phosphate (clindamycin phosphate); Coly-Mycin M (colistimethate sodium); and Vancocin HCl (vancomycin hydrochloride).

Penicillins include, e.g., Amoxil (amoxicillin); Augmentin (amoxicillin/clavulanate potassium); Bicillin C-R 900/300 (Penicillin G benzathine and Penicillin G procaine suspension); Bicillin C-R (Penicillin G benzathine and Penicillin G procaine suspension); Bicillin L-A (Penicillin G benzathine suspension); Geocillin (carbencillin indanyl sodium); Mezlin (sterile mezlocillin sodium); Omnipen (ampicillin); Pen-Vee K (penicillin V potassium); Pfizerpen (penicillin G potassium); Pipracil (piperacillin sodium); Spectrobid (bacampicillin HCl); Ticar (ticarcillin disodium); Timentin (ticarcillin disodium and clavulanate potassium); Unasyn (ampicillin sodium/sulbactam sodium); Zosyn (piperacillin sodium and tazobactam sodium); and Dicloxacillin Sodium.

Tetracyclines include, e.g., Achromycin V (tetracycline HCl); Declomycin (demeclocycline HCl); Dynacin (minocylcine HCl); Minocin (minocycline hydrochloride); Monodox (Doxycycline monohydrate capsules); Terramycin (oxytetracyline); Vectrin (minocycline hydrochloride); Vibramycin Calcium (doxycycline sodium); Vibramycin Hyclate (doxycycline hyclate); Vibramycin Monohydrate (doxycycline monohydrate); Vibra-Tabs (doxycycline hydrate); Declomycin (demeclocycline HCl); Vibramycin (doxycycline); Dynacin (Minocyline HCl); Terramycin (oxytetracycline HCl); Achromycin V capsules (tetracycline HCl); Lincomycins; and Cleocin HCl (clindamycin HCl).

Antifungals include, e.g., Abelcet (amphotericin B lipid complex); AmBisome (amphotericin B); Amphotec (amphotericin B cholesterol sulfate complex); Ancobon (flucytosine); Diflucan (fluconazole); Fulvicin P/G (ultramicrosize griseofulvin); Fulvicin P/G 165 and 330 (ultramicrosize griseofulvin); Grifulvin V (griseofulvin); Gris-PEG (griseofulvin ultramicrosize); Lamisil (terbinafine hydrochloride); Nizoral (ketoconazole); Amphotericin B; Lotrimin (clotrimazole); Dapsone tablets (dapsone); Diflucan (fluconazole); Monistat-Derm cream (miconazole); Mycostatin Cream (nystatin); and Sporanox (itraconazole).

Antimalarial agents include, e.g., Aralen hydrochloride (chloroquine HCl); Aralen phosphate (chloroquine phosphate); Daraprim (pyrimethamine); Lariam (mefloquine HCl); and Plaquenil (hydroxychloroquine sulfate).

Antituberculosis agents include, e.g., Capastat sulfate (capreomycin sulfate); Myambutol (ethambutol hydrochloride); Mycobutin (rifabutin capsules); Nydrazid (isoniazid injection); Paser (aminosalicylic acid); Priftin (rifapentine); Pyrazinamide tablets (pyrazinamide); Rifadin (rifampin capsules); Rifadin IV (rifampin for injection); Rifamate (rifampin and isoniazid); Rifater (rifampin, isoniazid and pyrazinamide); Seromycin (cycloserine capsules); Streptomycin Sulfate; Tice BCG (BCG vaccine); Cycloserine (seromycin capsules); Urised (Methenamine); and Trecator-SC (ethionamide tablets).

Antivirals include, e.g., Alferon N (interferon alfa-n3); Crixivan (indinavir sulfate); Cytovene (ganciclovir); Cytovene-IV (ganciclovir sodium); Epivir (lamivudine); Famvir (famciclovir); Flumadine (rimantadine HCl); Foscavir (foscamet sodium); Hivid (zalcitabine); Intron A (interferon alfa-2b); Invirase (saquinavir mesylate); Norvir (ritonavir); Rebetron combination therapy, which contains Rebetrol (ribavirin) and Intron A (inteferon alfa-2b); Rescriptor (delavirdine mesylate); Retrovir (ziduvudine); Retrovir IV (zidovudine); Symmetrel (amantadine hydrochloride); Synagis (palivizumab); Valtrex (valacyclovir HCl); Videx (didanosine); Viracept (nelfinavir mesylate); Viramune (nevirapine); Virazole (ribavirin); Vistide (cidofovir); Zerit (stavudine (d4T)); Symmetrel Syrup (amantadine HCl); Combivir Tablets (lamiduvine); and Zovirax (acyclovir).

Leprostatics include, e.g., Dapsone Tablets (dapsone).

Miscellaneous anti-infectives include, e.g., Daraprim (pyrimethamine); Flagyl 375 (metronidazole); Flagyl ER Tablets (metronidazole); Flagyl I.V. (metronidazole); Furoxone (furazolidone); Mepron (atovaquone); and Neutrexin (trimetrexate glucuronate).

Quinolones include, e.g., Cipro (ciprofloxacin HCl); Floxin (ofloxacin); Levaquin (levofloxacin); Mazaquin (lomefloxacin HCl); Noroxin (norfloxacin); Penetrex (enoxacin); Raxar (grepafloxacin HCl); Trovan (trovafloxacin mesylate); and Zagam (sparfloxacin).

Sulfonamides include, e.g., Bactrim (trimethoprim and sulfamethoxazole); Bactrim DS (trimethoprim and sulfamethoxazole double strength); Pediazole (erythromycin ethylsuccinate and sulfisaxazole acetyl); Septra (trimethoprim and sulfamethoxazole); Septra DS (trimethoprim and sulfamethoxazole); Co-Trimoxazole, Sulfadiazine, Bactrim I.V. Infusion (sulfamethoxazole); Sulfapyridine, and Pediazole (erythromycin ethylsuccinate and sulfisoxazole acetyl).

Urinary anti-infectives include, e.g., Furadantin (nitrofurantoin); Macrobid (nitrofurantoin monohydrate macrocrystals); Macrodantin (nitrofurantoin macrocrystals); Monurol Sachet (fosfomycin tromethamine); NegGram Caplets (nalidixic acid); Septra (trimethoprim and sulfamethoxazole); Septra DS (trimethoprim and sulfamethoxazole); Urised (a combination of the antiseptics methenamine, methylene blue, phenyl salicylate, benzoic acid and parasympatholytics (atropine sulfate) hyoscyamine); Urobiotic-250 Capsules (oxytetracycline HCl, sulfamethizole and phenazopyridine HCl); and Uroqid Acid No. 2 Tablets (methenamine mandelate).

Nasal antibiotics include, e.g., Bactroban (mupirocin).

Opthalmic antibiotics include, e.g., Chloromycetin opthalmic (chloramphenical); Cortisporin (neomycin and polymyxin β sulfates and hydrocortisone acetate cream); Ilotycin (erythromycin opthalmic ointment); NeoDecadron (neomycin sulfate—dexamethasone sodium phosphate); Polytrim (trimethoprim and polythyxin β sulfate opthalmic solution); Terra-Cortril (oxytetracycline HCl and hydrocortisone acetate); Terramycin (oxytetracycline); and TobraDex (tobramycin and dexamethasone opthalmic suspension and ointment).

Opthalmic antivirals include, e.g., Vira-A opthalmic ointment, (vidarabine).

Opthalmic quinalones include, e.g., Chibroxin (norfloxacin opthalmic solution); Ciloxan opthalmic solution, (Ciprofloxacin HCl); Ciloxan opthalmic ointment, (Ciprofloxacin HCl); and Ocuflox opthalmic solution (ofloxacin).

Opthalmic sulfonamides include, e.g., Blephamide opthalmic ointment (sulfacetamide sodium and prednisolone acetate); and Blephamide opthalmic suspension (sulfacetamide sodium and prednisolone acetate).

Skin and mucous membrane antibiotics include, e.g., A/T/S (erythromycin); Bactroban (mupirocin); Benzamycin (erythromycin-benzoyl peroxide topical gel); Betadine (povidone-iodine); Cleocin T (clindamycin phosphate topical solution); Clindets (clindamycin phosphate pledgets); Cortisporin (neomycin, polymyxin B sulfates and hydrocortisone acetate cream); Emgel (erythromycin); Erycette (erythromycin topical solution); Garamycin (gentamicin sulfate); Klaron (sodium sulfacetamide lotion); Mycostatin (nystatin cream); Theramycin Z (erythromycin topical solution); T-Stat (erythromycin); Chloromycetin (chloramphenicol opthalmic ointment); Cortisporin (neomycin and polymyxin B sulfates, bacitracin zinc and hydrocortisone opthalmic ointment); Ilotycin (erythromycin); NeoDecadron (neomycin sulfate-dexamethasone sodium phosphate); Polytrim (trimethoprim and polymyxin B sulfate); Terra-Cortril (oxytetracycline HCl and hydrocortisone acetate); Terramycin (oxytetracycline); and TobraDex (tobramycin and dexamethasone opthalmic suspension and ointment).

Skin and mucous membrane antifungals include, e.g., Exelderm (sulconazole nitrate); Fungizone (amphotericin B oral suspension); Lamisil (terbinafine hydrochloride cream); Loprox (ciclopiroxolamine); Lotrimin (clotrimazole); Lotrisone (clotrimazole and betamethasone diproprionate); Mentax (butenafine HCl); Monistat-Derm (miconazole nitrate); Mycelex (clotrimazole); Mycostatin (nystatin); Naftin (naftifine HCl); Nizoral (ketoconazole); Nystop (nystatin); Oxistat (oxiconazole nitrate); Selsun Rx (2.5% selenium sulfide lotion); and Spectazole (econazole nitrate).

Skin and mucous membrane antivirals include, e.g., Denavir (penciclovir cream); and Zovirax (acyclovir).

Skin and mucous membrane miscellaneous anti-infectives include, e.g., Benzashave (benzoyl peroxide); Betadine (povidone-iodine); Betasept (chlorhexidine gluconate); Cetaphil (soap substitute); Clorpactin WCS-90 (sodium oxychlorosene); Dapsone Tablets (dapsone); Desquam-E (benzoyl peroxide); Desquam-X (benzoyl peroxide); Hibiclens (chlorhexidine gluconate); Hibistat (chlorhexidine gluconate); Impregon (tetrachlorosalicylanilide 2%); MetroCream (metronidazole); MetroGel (metronidazole); Noritate (metronidazole); pHisoHex (hexachlorophene detergent cleanser); Sulfacet-R (sodium sulfacetamide 10% and sulfur 5%); Sulfamylon (matenide acetate); Triaz (benzoyl peroxide); and Vanoxide-HC (benzoyl peroxide hydrocortisone).

Skin and mucous membrane scabicides and pedulicides include, e.g., Acticin (permethrin); Elimite (permethrin); Eurax (crotamiton); and Lindane Lotion USP 1% (lindane).

Skin and mucous membrane antinepolasts include, e.g., Efudex (fluorouracil); and Fluoroplex (fluorouracil).

Nitrofurans include, e.g., Furadantin Oral Suspension (nitrofurantoin).

Oxazolidinones include, e.g., Zyvox (linezolid).

It is appreciated that those skilled in the art understand that the antibiotic useful in the present invention is the biologically active compound present in any of the antibiotic formulations disclosed above. For example, Azactam (aztreonam) is typically available as an injectable solution. The antibiotic, however, is (z)-2-[[[(2-amino-4-thiazolyl)[[(2-S,-3S)-2-methyl-4-oxo-1-sulfo-3-azetidinyl]carbamoyl]methylene]amino]oxy]-2-methyl propionic acid. Physician's Desk Reference (PDR), Medical Economics Company (Montvale, N.J.), (53rd Ed.), pp. 820-823, 1999.

Amikacin (amikacin sulfate) is commercially available from Elkins-Sinn and is D-Streptamine, O-3-amino-3-deoxy-α-D-glucopyranosyl-(1→6)-O-6-deoxy-α-D-glucopyranosyl-(1→4)]-N′-(4-amino-2hydroxy-1-oxobutyl)-2-deoxy-, (S)—, sulfate (1:2) (salt).

Garamycin (gentamicin sulfate) is commercially available from Schering.

Nebcin (tobramycin sulfate) is commercially available from Lilly and is O-3-amino-3-deoxy-α-D-glucopyranosyl-(1→4)-O-[2,6-diamino-2,3-6-trideoxy-α-D-ribo-hexopyranosyl-(1→6)]-2-deoxy-L-streptamine, sulfate (2:5) (salt).

Netromycin (netilmicin sulfate) is commercially available from Schering and is O-3-Deoxy-4-C-methyl-3-(methylamino)-β-L-ara-binopyranosyl(1→4)-O-[2,6-diamino-2,3,4,6-tetradeoxy-α-D-glycero-hex-4-enopyranosyl-(1→6)-2-deoxy-N³-ethyl-L-streptamine sulfate (2:5) salt.

Streptomycin Sulfate is commercially available from Pfizer and is D-Streptamine, O-2-deoxy-2-(methylamino)-α-L-glucopyranosyl-(1→2)-O-5-deoxy-3-C-formyl-α-L-lyxofuranosyl, (1→4)-N,N′-bis(aminoiminomethyl)-, sulfate (2:3) (salt).

TOBI (tobramycin) is commercially available from Pathogenesis Corporation and is O-3-amino-3-deoxy-α-D-glucopyranosyl-(1→4)-O-[2,6-diamino-2,3,6-trideoxy-α-D-ribo-hexopyranosyl-(1→6)]-2-deoxy-L-streptamine.

Azactam (aztreonam) is commercially available from Bristol-Myers Squibb and is (Z)-2-[[[(2-amino-4-thiazolyl)[[(2S,-3S)-2-methyl-4-oxo-1-sulfo-3-azetidinyl]carbamoyl]methylene]amino]oxy]-2-methylpropionic acid.

Cefotan (cefotetan) is commercially available from Zeneca and is the disodium salt of [6R-(6α,7α)]-7-[[[4-(2-amino-1-carboxy-2-oxoethylidene)-1-3, dithietan-2-yl]carbonyl]amino]-7-methoxy-3-[[(1-methyl-1H-tetrazol-5-yl)thio]methyl]-8-oxo-5-thia-1-azabicyclo[4.2.0]oct-2-ene-2-carboxylic acid.

Lorabid (loracarbef) is commercially available from Lilly and is (6R,7S)-7-[(R)-2-amino-2-phenylacetamido]-3-chloro-8-oxo-1-azabicyclo[4.2.0]oct-2-ene-2-carboxylic acid, monohydrate.

Mefoxin (cefoxitin) is commercially available from Merck and is sodium (6R,7S)-3-(hydroxymethyl)-7-methoxy-8-oxo-7-[2-(2-thienyl)acetamido]-5-thia-1-azabicylo[4.2.0]oct-2-ene-2-carboxylate carbamate (ester).

Merrem (meropenem) is commercially available from Zeneca and is (4R,5S,6S)-3-[(3S,5S)-5-(Dimethylcarbamoyl)-3-pyrrolidinyl]thiol]-6-[(1R)-1-hydroxyethyl]-4-methyl-7-oxo-1-azabicyclo[3.2.0]hept-2-ene-2-carboxylic acid trihydrate.

Primaxin (imipenem and cilastatin for injectable suspension) is commercially available from Merck and is (1) imipenem is N-formimidoylthienamycin monohydrate, chemical name is [5R-[5α,6α(R*)]]-6-(1-hydroxyethyl)-3-[[2-[(iminomethyl)amino]ethyl]thio]-7-oxo-1-azabicylco[3.2.0]hept-2-ene-2-carboxylic acid monohydrate, cilastatin sodium is [R-[R*,S*,-(Z)]]-7-[(2-amino-2-carboxyethyl)thio]-2-[[(2,2-dimethyl cyclopropyl)carbonyl]amino]-2-heptenoic acid, monosodium salt.

Ancef (cefazolin) is commercially available from SmithKline Beecham and is 3-{[(5-methyl-1,3,4-thiadiazol-2-yl)thio-methyl]}-8-oxo-7-[2-(1H-tetracol-1-yl)acetamido]-5-thia-1-azabicyclo[4.2.0]oct-2-ene-2-carboxylic acid.

Ceclor (cefaclor) is commercially available from Lilly and is 3-chloro-7-D-(2-phenylglycinamido)-3-cephem-4-carboxylic acid monohydrate; Cedax (ceftibuten) is commercially available from Schering and is (+)-(6R,7R)-7-[(Z)-2-(2-(2-amino-4-thiazoly)-4-carboxycrotonamido]-8-oxo-5-thia-1-azabicyclo[4.2.0]oct-2-ene-2-carboxylic acid, dihydrate.

Cefizox (ceftizoxime sodium) is commercially available from Fujisawa and is sodium salt of [6R-[6α,7β(Z)]]-7[[2,3,dihydro-2-imino-4-thiazolyl)(methoxyamino)acetyl]amino]-8-oxo-5-thia-1-azabicyclo[4.2.0]oct-2-ene-2-carboxyolic acid.

Cefobid (cefoperazone sodium) is commercially available from Pfizer and is sodium (6R,7R)-7[(R)-2-(4-ethyl-2,3,dioxo-1-piperazine carboxamido)-2-(p-hydroxyphenyl)-acetamido)-3-[[(1-methyl-1H-tetrazol-5-yl)thio]methyl]-8-oxo-5-thia-1-azabicyclo[4.2.0]oct-2-ene-2-carboxylate.

Ceftin (cefuroxime axetil) is commercially available from Glaxo Wellcome and is (RS)-1-hydroxyethyl(6R,7R)-7-[2-(2-furyl)glyoxylamido]-3-(hydroxyethyl)-(8-oxo-5-thia-1-aza-bicyclo[4.2.0]oct-2-ene-2-carboxylate, 7²(Z)-O-methyl-oxime), 1-acetate 3-carbamate.

Cefzil (cefprozil) is commercially available from Bristol-Myers Squibb and is (6R,7R)-7-[(R)-2-amino-2-(p-hydroxyphenyl)acetamido]-8-oxo-3-propenyl-5-thia-1-azabicyclo[4.2.0]oct-2-ene-2-carboxylic acid monohydrate.

Ceptaz (ceftazidime) is commercially available from Glaxo Wellcome and is 1-[[7-[[(2-amino-4-thiazolyl)[(1-carboxy-1-methylethoxy)imine]acetyl]amino]-2-carboxy-8-oxo-5-thia-1-azabicyclo[4.2.0]oct-2-en-3-yl]methyl]-, hydroxide, inner salt, [6R-[6α,7β(Z)]].

Claforan (cefotaxime) is commercially available from Hoescht Marion Roussel and is 7-[2-(2-amino-4-thiazolyl)glyoxylamido]-3-(hydroxy methyl)-8-oxo-5-thia-1-azabicyclo[4.2.0]oct-2-ene-2-carboxylate 7²(Z)-(O-methyloxime), acetate (ester).

Duricef (cefadroxil monohydrate) is commercially available from Bristol-Myers Squibb and is 5-Thia-1-azabicyclo[4.2.0]oct-2-ene-2-carboxylic acid, 7-[[amino(4-hydroxyphenyl)acetyl]amino]-3-methyl-8-oxo, -monohydrate, [6R-[6α,7β(R*)]]—.

Fortaz (ceftazidime) is commercially available from Glaxo Wellcome and is 1-[[7-[[(2-amino-4-thiazolyl)[1-carboxy-1-methylethoxy)imino]acetyl]amino]-2-carboxy-8-oxo-5-thia-1-azabicyclo[4.2.0]oct-2-en-3-yl]methyl]-, hydroxide, inner salt [6R-[6α,7β(Z)]].

Keflex (cephalexin) is commercially available from Dista and is 7-(D-α-Amino-α-phenyl acetamido)-3-methyl-3-cephem-4-carboxylic acid monohydrate.

Keftab (cephalexin HCl) is commercially available from Dura and is 7-(D-2-Amino-2-phenylacetamido)-3-methyl-3-cephem-4-carboxylic acid hydrochloride monohydrate.

Kefurox (cefuroxime) is commercially available from Lilly and is the sodium salt of (6R.7R)3-carbamoyloxymethyl-7-[Z-2-methoxyimino-2-(fur-2-yl)acetamiodo]ceph-3-em-4-carboxylate.

Kefzol (cefazolin) is commercially available from Lilly and is the sodium salt of 3-{[(5-methyl-1,3,4-thiadiazol-2-yl)thio]methyl}-8-oxo-7-[2-(1H-tetrazol-1-yl)acetamido]-5-thia-1-azabicyclo[4.2.0]oct-2-ene-2-caboxylic acid.

Mandol (cefamandole nafate) is commercially available from Lilly and is 5-thia-1-azabicyclo[4.2.0]oct-2-ene-2-carboxylic acid, 7-[[(formyloxy)phenyl acetyl]amino]-3-[[(1-methyl-1H-tetrazol-5-yl)thio]methyl]-8-oxo-, mono-sodium salt, [6R-[6α-7β(R*)]].

Maxipime (cefepime HCl) is commercially available from Bristol-Myers Squibb and is 1-[[6R,7R)-7-[2-amino-4-thiazolyl)-glyoxylamido]-2-carboxy-8-oxo-5-thia-1-azabicyclo-[4.2.0]oct-2-en-3-yl]methyl]-1-methylpyrrolidinium chloride, 7²-(Z)-(O-methyloxime), monohydrochloride, monohyrate.

Monocid (cefonicid sodium) is commercially available from SmithKline Beecham and is 5-Thia-1-azabicyclo[4.2.0]oct-2-ene-2-carboxylic acid, 7-[(hydroxyphenyl-acetyl)-amino]-8-oxo-3-[[1-(sulfomethyl)-1H-tetrazol-5-yl]thio]methyl]-disodium salt, [6R-[6α,7β(R*)]].

Omnicef (cefdinir) is commercially available from Parke Davis and is [6R-[6α,7β(Z)]]-7-[[(2-amino-4-thiazolyl)-(hydroxyimino)acetyl]amino]-3-ethenyl-8-oxo-5-thia-1-azabicyclo[4.2.0]oct-2-ene-2-carboxylic acid.

Rocephin (ceftriaxone) is commercially available from Roche Laboratories and is (6R,7R)-7-[2-(2-Amino-4-thiazolyl)glyoxylamido]-8-oxo-3-[[(1,2,5,6-tetrahydro-2-methyl-5,6-dioxo-as-triazin-3-yl)thio]methyl]-5-thia-1-azabicyclo[4.2.0]oct-2-ene-2-carboxylic acid, 7²-(Z)-O-methyloxime), disodium salt, sesquaterhydrate.

Suprax (cefixime) is commercially available from Lederle Laboratories and is (6R,7R)-7-[2-(2-Amino-4-thiazolyl)glyoxylamido]-8-oxo-3-vinyl-5-thia-1-azabicyclo[4.2.0]oct-2-ene-2-carboxylic acid, 7²-(Z)-[O-(carboxymethyl)oxime]trihydrate.

Tazicef (ceftazidime) is commercially available from SmithKline Beecham and is pentahydrate of Pyridinium, 1-[[7-[[2-amino-4-thiazolyl)[(1-carboxy-1-methylethoxy)imino]acetyl]amino]-2-carboxy-8-oxo-5-thia-1-azabicyclo(4.2.0)oct-2-en-3-yl]methyl]-hydroxide, inner salt, [6R, -[6α,7β(Z)]].

Tazidime (ceftazidime) is commercially available from Lilly and is pentahydrate of Pyridinium, 1-[[7-[[2-amino-4-thiazolyl)[(1-carboxy-1-methylethoxy)imino]acetyl]amino]-2-corboxy-8-oxo-5-thia-1-azabicyclo(4.2.0)oct-2-en-3-yl]methyl]-hydroxide, inner salt, [6R,-[6α,7β(Z)]].

Vantin (cefpodoxime proxetil) is commercially available from Pharmacia & Upjohn and is (RS)-1(isopropoxycarbonyloxy)ethyl(+)-(6R,7R)-7-[2-(2-amino-4-thiazolyl)-2-{(Z)methoxyimino}acetamido]-3-methoxymethyl-8-oxo-5-thia-1-azabicyclo[4.2.0]oct-2-ene-2-carboxylate.

Zinacef (cefuroxime) is commercially available from Glaxo Wellcome and is sodium salt of (6R,7R)-3-carbamoyloxymethyl-7-[Z-2-methoxy-imino-2-fur-2-yl)acetamido]ceph-3-em-4-carboxylate.

Biaxin (clarithromycin) is commercially available from Abbott and is 6-O-methylerythromycin.

Dynabac (dirithromycin) is commercially available from Sanofi and is (9S)-9-Deox-11-deoxy-9,11-[imino[(1R)-2-(2-methoxyethoxy)-ethylidene]oxy]erythromycin.

E.E.S. 200 (Erythromycin Ethylsuccinate) is commercially available from Abbott and is erythromycin 2′-(ethylsuccinate).

E.E.S. 400 (Erythromycin Ethylsuccinate) is commercially available from Abbott and is erythromycin 2′-(ethylsuccinate).

Ery-Ped 200 (Erythromycin Ethylsuccinate) is commercially available from Abbott and is erythromycin 2′-(ethylsuccinate).

EryPed 400 (Erythromycin Ethylsuccinate) is commercially available from Abbott and is erythromycin 2′-(ethylsuccinate).

Ery-Tab (Erythromycin delayed-release tablets) is commercially available from Abbott and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-β-D-xylo-hex-opyranosyl]oxy]oxacyclotetradecane-2,10-dione.

Erythrocin Stearate (Erythromycin stearate) is commercially available from Abbott and is the stearate salt of (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-β-D-xylo-hex-opyranosyl]oxy]oxacyclotetradecane-2,10-dione.

Ilosone (erythromycin estolate) is commercially available from Dista and is erythromycin 2′-propionate, dodecyl sulfate.

PCE Dispertab (erythromycin particles in tablets) is commercially available from Abbott and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-β-D-xylo-hex-opyranosyl]oxy]oxacyclotetradecane-2,10-dione.

Pediazole (erythromycin ethylsuccinate and sulfisoxazole acetyl for oral suspension) is commercially available from Ross Products and is 2′-ethylsuccinyl ester of erythromycin (erythromycin ethylsuccinate) and N-(3,4-dimethyl-5-isoxazolyl)-N-sulfanilylacetamide (sulfisoxazole acetyl).

Tao (troleandomycin) is commercially available from Pfizer and is the synthetically derived acetylated ester of oleandomycin.

Zithromax (azithromycin) is commercially available from Pfizer and is (2R,3S,4R,5R,8R,10R,11R,12S,13S,14R)-13-[(2,6-dideoxy-3-C-methyl-3-O-methyl-α-L-ribo-hexopyranosyl)oxy]-2-ethyl-3,4,10-trihydroxy-3,5,6,8,10,12,14-heptamethyl-11-[[3,4,6-trideoxy-3-(dimethylamino)-β-D-xylo-hexopyranosyl]oxy]-1-oxa-6-azacyclopentadecan-15-one.

Erythromycin, which is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-β-D-xylo-hex-opyranosyl]oxy]oxacyclotetradecane-2,10-dione.

Cleocin HCl (clindamycin hydrochloride) is commercially available from Pharmacia & Upjohn and is the hydrated hydrochloride salt of clindamycin, a semisynthetic antibiotic produced by a 7(S)-chlorosubstitution of the (7R) hydroxyl group of lincomycin.

Cleocin Phosphate (clindamycin phosphate) is commercially available from Pharmacia & Upjohn and is L-threo-α-D-galacto-Octopyranoside, methyl 7 chloro-6,7,8,-trideoxy-6-[[(1-methyl-4-propyl-2-pyrrolidinyl) carbonyl]amino]-1-thio-, 2-(dihydrogen phosphate), (2S-trans)-.

Coly-Mycin M (colistimethate sodium) is commercially available from Monarch.

Vancocin HCl (vancomycin hydrochloride) is commercially available from Lilly.

Amoxil (amoxicillin) is commercially available from SmithKline Beecham and is (2S,5R,6R)-6-[(R)-(−)-2-amino-2-(p-hydroxyphenyl) acetamido]-3,3-dimethyl-7-oxo-4-thia-1-azabicyclo[3.2.0]-heptane-2-carboxylic acid trihydrate.

Augmentin (amoxicillin/clavulanate potassium) is commercially available from SmithKline Beecham and is (2S,5R,6R)-6-[(R)-(−)-2-amino-2-(p-hydroxy phenyl)acetamido]-3,3-dimethyl-7-oxo-4-thia-1-azabicyclo[3.2.0]-heptane-2-carboxylic acid trihydrate (amoxicillin) and potassium (Z)-(2R,5R)-3-(2-hydroxyethylidene)-7-oxo-4-oxa-1-azabicyclo[3.2.0]-heptane-2-carboxylate (clavulanate potassium).

Bicillin C-R 900/300 (Penicillin G benzathine and Penicillin G procaine suspension) is commercially available from Wyeth-Ayerst and is (2S,5R,6R)-3,3-Dimethyl-7-oxo-6-(2-phenylacetamido)-4-thia-1-azabicyclo [3.2.0]heptane-2-carboxylic acid compound with N,N′-dibenzylethylenediamine (2:1), tetrahydrate (Penicillin G benzathine) and (2S,5R,6R)-3,3-Dimethyl-7-oxo-6-(2-phenylacetamido)-4-thia-1-azabicyclo[3.2.0]heptane-2-carboxylic acid compound with 2-(diethylamino) ethyl p-amino benzoate compound (1:1) monohydrate (Penicillin G procaine).

Bicillin C-R (Penicillin G benzathine and Penicillin G procaine suspension) is commercially available from Wyeth-Ayerst and is (2S,5R,6R)-3,3-Dimethyl-7-oxo-6-(2-phenylacetamido)-4-thia-1-azabicyclo[3.2.0]heptane-2-carboxylic acid compound with N,N′-dibenzylethylenediamine (2:1), tetrahydrate (Penicillin G benzathine) and (2S,5R,6R)-3,3-Dimethyl-7-oxo-6-(2-phenylacetamido)-4-thia-1-azabicyclo[3.2.0]heptane-2-carboxylic acid compound with 2-(diethylamino) ethyl p-amino benzoate compound (1:1) monohydrate (Penicillin G procaine).

Bicillin L-A (Penicillin G benzathine suspension) is commercially available from Wyeth-Ayerst and is (2S,5R,6R)-3,3-Dimethyl-7-oxo-6-(2-phenylacetamido)-4-thia-1-azabicyclo[3.2.0]heptane-2-carboxylic acid compound with N,N′-dibenzylethylenediamine (2:1), tetrahydrate.

Geocillin (carbencillin indanyl sodium) is commercially available from Pfizer and is 1-(5-Indanyl)-N-(2-carboxy-3-3-dimethyl-7-oxo-4-thia-1-azabicyclo[3.2.0]hept-6-yl)-2-phenylmalonamate monsodium salt.

Mezlin (sterile mezlocillin sodium) is commercially available from Bayer and is the monohydrate sodium salt of 6-{D-2[3[(methyl-sulfonyl)-2-oxo-imidazolidine-1-carboxamido]-2-phenyl acetamido}penicillanic acid.

Omnipen (ampicillin) is commercially available from Wyeth-Ayerst and is (2S,5R,6R)-6-[(R)-2-Amino-2-phenylacetamido]-3,3-dimethyl-7-oxo-4-thia-1-aza-bicyclo[3.2.0]heptane-2-carboxylic acid.

Pen-Vee K (penicillin V potassium) is commercially available from Wyeth-Ayerst and is the potassium salt of the phenoxymethyl analog of penicillin G.

Pfizerpen (penicillin G potassium) is commercially available from Pfizer and is monopotassium 3,3-dimethyl-7-oxo-6-(2-phenylacetamido)-4-thia-1-azabicyclo(3.2.0)heptane-2-carboxylate.

Pipracil (piperacillin sodium) is commercially available from Lederle and is 4-thia-1-azabicyclo[3.2.0]heptane-2-carboxylic acid, 6-[[[[(4-ethyl-2-3-dioxo-1-piperazinyl)carbonyl]amino]phenylacetyl]amino]-3,3-dimethyl-7-oxo-, monosodium salt, [2S-[2α,5α,6β(S*)]].

Spectrobid (bacampicillin HCl) is commercially available from Pfizer and is 1′-ethoxycarbonyloxyethyl-6-(D-α aminophenylacetamide)-penicillate hydrochloride.

Ticar (ticarcillin disodium) is commercially available from SmithKline Beecham and is N-(2-carboxy-3,3-dimethyl-7-oxo-4-thia-1-azabicyclo[3.2.0]hept-6-yl)-3-thiophenemalonamic acid disodium salt.

Timentin (ticarcillin disodium and clavulanate potassium) is commercially available from SmithKline Beecham and is N-(2-carboxy-3,3-dimethyl-7-oxo-4-thia-1-azabicyclo[3.2.0]hept-6-yl)-3-thiophenemalonamic acid disodium salt (ticarcillin disodium) and potassium (Z)-(2R,5R)-3-(2-hydroxyethylidene)-7-oxo-4-oxa-1-azabicyclo[3.2.0]heptane-2-carboxylate (clavulanate potassium).

Unasyn (ampicillin sodium/sulbactam sodium) is commercially available from Pfizer and is monosodium (2S,5R,6R)-6-[(R)-2-Amino-2-phenyl acetamido]-3,3-dimethyl-7-oxo-4-thia-1-aza-bicyclo[3.2.0]heptane-2-carboxylate (amipicillin sodium), and sodium penicillate sulfone; sodium (2S,5R)-3,3-dimethyl-7-oxo-4-thia-1-azabicyclo[3.2.0]heptane-2-carboxylate 4,4-dioxide (sulbactam sodium).

Zosyn (piperacillin sodium and tazobactam sodium) is commercially available from Lederle and is sodium (2S,5R,6R)-6[(R)-2-(4-ethyl-2,3-dioxo-1-piperazine-carboxamido)-2-phenylacetamido]-3,3-dimethyl-7-oxo-4-Thia-1-azabicylco-[3.2.0]heptane-2-carboxylate (piperacillin sodium), and sodium (2S,3S,5R)-3-methyl-7-oxo-3-(1H-1,2,3-triazol-1-ylmethyl)-4-thia-1-azabicyclo[3.2.0]heptane-2-carboxylate-4,4-dioxide (tazobactam sodium).

Dicloxacillin Sodium is monosodium (2S,5R,6R)-6-(3-(2,6-dichlorophenyl)5-methyl-4-isoxazolecarboxamido]-3,3-dimethyl-7-OXO-4-thia-1-azabicyclo[3.2.0]heptane-2-carboxylate monohydrate.

Achromycin V (tetracycline HCl) is commercially available from Lederle and is the monohydrochloride of [4S-(4α,4aα,5aα,6β,12aα)]-4-(Dimethylamino)-1,4,4a,5,5a,6,11,12a-octahydro-3,6,10,12,12a-pentahydroxy-6-methyl-1,11-dioxo-2-naphthacenecarboxamide.

Declomycin (demeclocycline HCl) is commercially available from Lederle Laboratories and is 7-chloro-4-dimethylamino)-1,4,4a,5,5a,6,11,12a-octahydro-3,6,10,12,12a-pentahydroxy-1,11-dioxo-2-naphthacenecarboxamide monohydrochloride.

Dynacin (minocylcine HCl) is commercially available from Medicis and is [4S-(4α,4aα,5aα,12aα)]-4,7-bis(dimethylamino)-1,4,4a,5,5a,6,11,12a-octahydro-3,10,12,12a-tetrahydroxy-1,11-dioxo-2-napthacene carboxamide monochloride.

Minocin (minocycline hydrochloride) is commercially available from Lederle Laboratories and is [4S-(4α,4aα,5aα,12aα)]-4,7-bis (dimethylamino)-1,4,4a,5,5a,6,11,12a-octahydro-3,10,12,12a-tetrahydroxy-1,11-dioxo-2-napthacene carboxamide monochloride.

Monodox (Doxycycline monohydrate capsules) is commercially available from Oclassen and is α-6-deoxy-5-oxytetracycline.

Terramycin (oxytetracyline) is commercially available from Pfizer.

Vectrin (minocycline hydrochloride) is commercially available from Warner Chilcott Professional Products and is [4S-(4α,4aα,5aα,12aα)]-4,7-bis(dimethylamino)-1,4,4a,5,5a,6,11,12a-octahydro-3,10,12,12a-tetrahydroxy-1,11-dioxo-2-napthacene carboxamide monochloride.

Vibramycin Calcium (doxycycline sodium) is commercially available from Pfizer and is 4-(Dimethylamino)-1,4,4a,5,5a,6,11,12a-octahydro-3,5,10,12,12a-pentahydroxy-6-methyl-1,11-dioxo-2-napthacene-carboxamide monohydrate.

Vibramycin Hyclate (doxycycline hyclate) is commercially available from Pfizer and is α-6-deoxy-5-oxytetracycline.

Vibramycin Monohydrate (doxycycline monohydrate) is commercially available from Pfizer and is 4-(Dimethylamino)-1,4,4a,5,5a,6,11,12a-octahydro-3,5,10,12,12a-pentahydroxy-6-methyl-1,11-dioxo-2-napthacene-carboxamide monohydrate.

Vibra-Tabs (doxycycline hydrate) is commercially available from Pfizer and is α-6-deoxy-5-oxytetracycline.

Vibramycin (doxycycline) is commercially available from Pfizer and is 4-(Dimethylamino)-1,4,4a,5,5a,6,11,12a-octahydro-3,5,10,12,12a-pentahydroxy-6-methyl-1,11-dioxo-2-napthacene-carboxamide monohydrate.

Lincomycins is monosodium (2S,5R,6R)-6-(3-(2,6-dichlorophenyl)5-methyl-4-isoxazolecarboxamido]-3,3-dimethyl-7-OXO-4-thia-1-azabicyclo[3.2.0]heptane-2-carboxylate monohydrate.

Cleocin HCl (clindamycin HCl) is commercially available from Pharmacia & Upjohn and is Methyl 7-chloro-6,7,8-trideoxy-6-(1-methyl-trans-4-propyl-L-2-pyrrolidine carboxamido)-1-thio-L-threo-α-D-galacto-octopyranoside monohydrochloride.

Abelcet (amphotericin B lipid complex) is commercially available from Libosome Company, Inc. and is [1R-(1R*,3S*,5R*,6R*,9R*,11R*,15S*,16R*,17R*,18S*,19E,21E,23E,25E,27E,29E,31E,33R*,35S*,36R*,37S*)]-33-[(3-amino-3,6-dideoxy-O-D-mannopyranosyl)-oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid.

AmBisome (amphotericin B) is commercially available from Fujisawa Healthcare and is [1R-(1R*,3S*,5R*,6R*,9R*,lI R*,15S*,16R*,17R*,18S*, 19E,21E,23E,25E,27E,29E,31E,33R*,35S*,36R*,37S*)]-33-[(3-amino-3,6-dideoxy-β-D-mannopyranosyl)-oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid.

Amphotec (amphotericin B cholesterol sulfate complex) is commercially available from Sequus Pharmaceuticals, Inc. and is [1R-(1R*,3S*,5R*,6R*,9R*,11R*,15S*,16R*,17R*,18S*,19E,21E,23E,25E,27E,29E,31E,33R*,35S*, 36R*,37S*)]-33-[(3-amino-3,6-dideoxy-β-D-mannopyranosyl)-oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid.

Ancobon (flucytosine) is commercially available from ICN Pharmaceuticals and is 5-fluorocytosine.

Diflucan (fluconazole) is commercially available from Pfizer Inc. and is 2,4-difluoro-α-α′-bis(1H-1,2,4-triazol-1-ylmethyl)benzyl alcohol.

Fulvicin P/G (ultramicrosize griseofulvin) is commercially available from Schering.

Fulvicin P/G 165 and 330 (ultramicrosize griseofulvin) is commercially available from Schering.

Grifulvin V (griseofulvin) is commercially available from Ortho Dermatological.

Gris-PEG (griseofulvin ultramicrosize) is commercially available from Allergan.

Lamisil (terbinafine hydrochloride) is commercially available from Novartis and is (E)-N-(6,6-dimethyl-2-hepten-4-ynyl)-N-methyl-1-naphthalenemethanamine hydrochloride.

Nizoral (ketoconazole) is commercially available from Janssen and is cis 1-acetyl-4-[4-[[2-(2,4-di-chlorophenyl)-2-(1H-imidazol-1-ylmethyl)-1,3-dioxolan-4-yl]methoxy]phenyl]piperazine.

Amphotericin B is [1R-(1R*,3S*,5R*,6R*,9R*,11R*,15S*,16R*,17R*,18S*,19E,21E,23E,25E,27E,29E,31E,33R*,35S*,36R*,37S*)]-33-[(3-amino-3,6-dideoxy-β-D-mannopyranosyl)-oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid.

Lotrimin (clotrimazole) is commercially available from Schering and is 1-(O-Chloro-α,α-diphenyl benzyl)imidazole.

Dapsone tablets (dapsone) is commercially available from Jacobus and is 4,4′-diaminodiphenyl-sulfone (DDS).

Diflucan (fluconazole) is commercially available from Pfizer and is 2,4-difluoro-α-α′-bis(1H-1,2,4-triazol-1-ylmethyl) benzyl alcohol.

Monistat-Derm cream (miconazole) is commercially available from Ortho Dermatological and is 1-[2,4-dichloro-β-{(2,4-dichlorobenzyl)oxy}phenethyl]imidazole mononitrate.

Mycostatin Cream (nystatin) is commercially available from Westwood-Squibb.

Sporanox (itraconazole) is commercially available from Janssen Pharmaceutical and is (±)-1-[(R*)-sec-butyl]-4-[p-[[2R*,4S*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ²-1,2,4,triazolin-5-one mixture with (±)-1-[(R*)-sec-butyl]-4-[p-[[2R*,4S*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ²-1,2,4,triazolin-5-one or (±)-1-[(RS)-sec-butyl]-4-[p-[4-[p-[[(2R,4S)-2-(2,4-dichlorophenyl)-2-(1H-1,2,3,4-triazol-1-ylmethy)1,3-dioxolan-4-yl]methoxy]phenyl]-1-piperazinyl]phenyl]-Δ²-1,2,4,triazolin-5-one.

Aralen hydrochloride (chloroquine HCl) is commercially available from Sanofi Pharmaceuticals and is 7-(chloro-4-[[4-diethylamino)-1-methyl butyl]amino]-quinoline dihydrochloride.

Aralen phosphate (chloroquine phosphate) is commercially available from Sanofi Pharmaceuticals and is 7-(chloro-4-[[4-diethylamino)-1-methyl butyl]amino]-quinoline phosphate (1:2).

Daraprim (pyrimethamine) is commercially available from Glaxo Wellcome and is 5-(4-chlorophenyl)-6-ethyl-2,4-pyrimidinediamine.

Lariam (mefloquine HCl) is commercially available from Roche Laboratories and is (R*,S*)-(±)-α-2-piperidinyl-2,8-bis(trifluoromethyl)-4-quinoline methanol hydrochloride.

Plaquenil (hydroxychloroquine sulfate) is commercially available from Sanofi Pharmaceuticals and is 2-[[4-[7-chloro-4-quinolyl)amino]pentyl]ethylamino]ethanol sulfate (1:1).

Capastat sulfate (capreomycin sulfate) is commercially available from Dura Pharmaceuticals.

Myambutol (ethambutol hydrochloride) is commercially available from Lederle Laboratories.

Mycobutin (rifabutin capsules) is commercially available from Pharmacia & Upjohn and is 1′,4-didehydro-1-deoxy-1,4-dihydro-5′-(2-methylpropyl)-1-oxorifamycin XIV or (9S,12E,14S,15R,16S,17R,18R,19R,20S,21S,22E,24Z)-6,16,18,20-tetrahydroxy-1-1′-isobutyl-14-methoxy-7,9,15,17,19,21,25-heptamethyl-spiro[9,4-(epoxypentadeca[1,11,13]trienimino)-2H-furo[2′, 3′:7,8]naphth[1,2-d]imidazole-2,4′-piperidine]-5,10,26-(3H,9H)-trione-16-acetate.

Nydrazid (isoniazid injection) is commercially available from Apothecon.

Paser (aminosalicylic acid) is commercially available from Jacobus and is 4-amino-2-hydroxy benzoic acid.

Priftin (rifapentine) is commercially available from Hoechst Marion Roussel and is rifamycin 3-[[(4-cyclo-pentyl-1-piperazinyl)imino]methyl]or 3[N-(4-cyclopentyl-1-piperazinyl)-formimyidoyl]-2,7-(epoxypentadeca[1,11,13]trienimino)naptho[2,1-b]furan-1,11(2H)-dione21-acetate.

Pyrazinamide tablets (pyrazinamide) is commercially available from Lederle Laboratories and is the pyrazine analogue of nicotinamide.

Rifadin (rifampin capsules) is commercially available from Hoechst Marion Roussel and is 3-[[(4-methyl-1-piperazinyl)imino]methyl]rifamycin or 5,6,9,17,19,21-hexahydroxy-23-methoxy-2,4,12,16,20,22-heptamethyl-8-[N-methyl-1-piperazinyl)formimidoyl]-2,7-(epoxy pentadeca[1,11,13]trienimino)naptho[2,1-b]furan-1,11(2H)-dione 21-acetate.

Rifadin IV (rifampin for injection) is commercially available from Hoechst Marion Roussel and is 3-[[3-(4-methyl-1-piperazinyl)formimidoyl]-2,7-(epoxy pentadeca[1,11,13]trienimino)naphtho[2,1-b]furan-1,11(2H)-dione 21-acetate.

Rifamate (rifampin and isoniazid) is commerically available from Hoechst Marion Roussel and is 3-(4-methyl-1-piperazinyliminomethyl)rifamycin SV (rifampin) and hydrazide of isonicotinic acid (isoniazid).

Rifater (rifampin, isoniazid and pyrazinamide) is commercially available from Hoechst Marion Roussel and is 3-(4-methyl-1-piperazinyliminomethyl)rifamycin SV (rifampin), hydrazide of isonicotinic acid (isoniazid), and pyrazine analogue of nicotinamide (pyrazinamide).

Seromycin (cycloserine capsules) is commercially available from Dura Pharmaceuticals and is 3-isoxazolidinone, 4-amino-, (R)-.

Streptomycin Sulfate is commercially available from Pfizer and is O-2-deoxy-2-(methylamino)-α-L-glyucopyransoyl-(1-2)-O-5-deoxy-3-C-formyl-α-L-lyxofuranosyl-(1→4)-N-N′-bis(aminoiminomethyl)-, sulfate (2:3) salt.

Tice BCG (BCG vaccine) is commercially available from Organon and is attenuated live Mycobacterium bovis strains Bacillus of Calmette and Guerin.

Cycloserine (seromycin capsules) is commercially available from Dura Pharmaceuticals and is 3-isoxazolidinone, 4-amino-, (R)-.

Nydrazid (Isoniazid) is commercially available from Apothecon and is the hydrazide of isonicotinic acid.

Urised (Methenamine) is commercially available from Poly Medica.

Trecator-SC (ethionamide tablets) is commercially available from Wyeth-Ayerst and is 2-ethylthioisonicotinamide.

Alferon N (interferon alfa-n3) is commercially available from Interferon Sciences and is interferon alfa-n3 (human leukocyte derived).

Crixivan (indinavir sulfate) is commercially available from Merck & Co., Inc. and is [1(1S,2R),5(S)]-2,3,5-trideoxy-N-(2,3-dihydro-2-hydroxy-1H-inden-1-yl)-5-[2-[[1,1-dimethylethyl)amino]carbonyl]-4-(3-pyridinyl-methyl)-1-piperazinyl]-2-(phenylmethyl)-D-erythropentonamide sulfate (1:1).

Cytovene (ganciclovir) is commercially available from Roche and is 9-[[2-hydroxy-1(hydroxymethyl)ethoxy]methyl]guanine.

Cytovene-IV (ganciclovir sodium) is commercially available from Roche and is 9-[[2-hydroxy-1(hydroxymethyl)ethoxy]methyl]guanine.

Epivir (lamivudine) is commercially available from Glaxo Wellcome and is (2R, cis)-4-amino-1-(2-hydroxymethyl-1,3-oxathiolan-5-yl)-1H)-pyrimidin-2-one.

Famvir (famciclovir) is commercially available from SmithKline Beecham and is 2-[2-(2-amino-9H-purin-9-yl)ethyl]-1,3-propanediol diacetate.

Flumadine (rimantadine HCl) is commercially available from Forest and is alpha-methyltricyclo-[3.3.1.1/3.7]decane-1-methanamine hydrochloride.

Foscavir (foscarnet sodium) is commercially available from Astra and is phosphonoformic acid, trisodium salt.

Hivid (zalcitabine) is commercially available from Roche and is 4-amino-1-beta-D-2′,3′, dideoxyribofuranosyl-2-(1H)-pyrimidone or 2′,3′-dideoxycytidine.

Intron A (interferon alfa-2b) is commercially available from Schering.

Invirase (saquinavir mesylate) is commercially available from Roche Labs and is N-tert-butyl-decahydro-2-[2(R)-hydroxy-4-phenyl-3(S)-[[N-(2-quinolylcarbonyl)-L-asparaginyl]amino]butyl-(4aS,8aS)-isoquinoline-3(S)-carboxamide methanesulfonate.

Norvir (ritonavir) is commercially available from Abbott and is 10-Hydroxy-2-methyl-5-(1-methylethyl)-1-[2-(1-methylethyl)-4-thiazolyl]-3,6-dioxo-8,11-bis(phenylmethyl)-2,4,7,12-tetraazatridencan-B-oic acid, 5-thiazolyl methyl ester [5S-(5R*,8R*,10R*,11R*)].

Rebetron combination therapy, which contains Rebetrol (ribavirin which is 1-β-D-ribofuranosyl-1H-1,2,4-triazole-3-carboxamide) and Intron A (inteferon alfa-2b), is commercially available from Schering.

Rescriptor (delavirdine mesylate) is commercially available from Pharmacia & Upjohn and is pieperazine, 1-[3-[(1-methylethyl) amino]-2-pyridinyl]-4-[[5(methylsulfonyl)-amino]-1H-indol-2-yl]carbonyl], monomethanesulfonate.

Retrovir (ziduvudine) is commerically available from Glaxo Wellcome and is 3′-azido-3′-deoxythymidine.

Retrovir IV (zidovudine) is commercially available from Glaxo-Wellcome and is 3′-azido-3′-deoxythymidine.

Symmetrel (amantadine hydrochloride) is commercially available from Endo Pharmaceuticals and is 1-adamantanamine hydrochloride.

Synagis (palivizumab) is commercially available from Medrmmune Inc. and is humanized monoclonal antibody (IgG1ₖ).

Valtrex (valacyclovir HCl) is commercially available from Glaxo Wellcome and is L-valine, 2-[(2-amino-1,6-dihydro-6-oxo-9H-purin-9-yl) methoxy]ethyl ester, monohydrochloride.

Videx (didanosine) is commercially available from Bristol-Myers Squibb Oncology/Immunology and is 2′,3′-di-deoxyinosine.

Viracept (nelfinavir mesylate) is commercially available from Agouron and is [3S-[2(2S*,3S*), 3α,4aβ,8aβ]]-N-(1,1-dimethylethyl)decahydro-2-[2-hydroxy-3-[(3-hydroxy-2-methylbenzoyl)amino]-4-(phenylthio)butyl]-3-isoquinolinecarboxcamide mono-methanesulfonate (salt).

Viramune (nevirapine) is commercially available from Roxane and is 11-cyclopropyl-5,11-dihydro-4-methyl-6H-dipyrido[3,2-b:2′,3′-][1,4]diazepin-6-one.

Virazole (ribavirin) is commercially available from ICN and is 1-beta-D-ribofuranosyl-1H-1,2,4-triazole-3-carboxamide.

Vistide (cidofovir) is commercially available from Gilead Sciences and is 1-[(S)-3-hydroxy-2-(phosphonomethoxy)propyl]cytosine dihydrate (HPMPC).

Zerit (stavudine (d4T)) is commercially available from Bristol-Myers Squibb Oncology/Immunology and is 2′,3′-didehydro-3′deoxythymidine.

Symmetrel Syrup (amantadine HCl) is commercially available from Endo Labs and is 1-adamantanamine hydrochloride.

Combivir Tablets (lamiduvine) is commercially available from Glaxo Wellcome and is 2′,3′-didehydro-3′-deoxythymidine.

Zovirax (acyclovir) is commercially available from Glaxo Wellcome and is 2-amino-1,9-dehydro-9-[(2-hydroxyethyoxy)methyl]-6H-purin-6-one.

Dapsone Tablets (dapsone) is commercially available from Jacobus and is 4,4′-diaminodiphenylsulfone (DDS).

Daraprim (pyrimethamine) is commercially available from Glaxo Wellcome and is 5-(4-chlorophenyl)-6-ethyl-2,4-pyrimidinediamine.

Flagyl 375 (metronidazole) is commercially available from Searle and is 2-Methyl-5-nitro-imidazole-1-ethanol.

Flagyl ER Tablets (metronidazole) is commercially available from Searle and is 2-Methyl-5-nitro-imidazole-1-ethanol.

Flagyl I.V. (metronidazole) is commercially available from SCS and is 2-Methyl-5-nitro-imidazole-1-ethanol.

Furoxone (furazolidone) is commercially available from Roberts and is 3-(5-nitrofurfuryliden-amino)-2-oxazolidinone.

Mepron (atovaquone) is commercially available from Glaxo Wellcome and is trans-2-[4-(4-chlorophenyl)cyclohexyl]-3-hydroxy-1,4-naphthalenedione.

Neutrexin (trimetrexate glucuronate) is commercially available from U.S. Bioscience and is 2,4-diamino-5-methyl-6-[(3,4,5-trimethoxyanilino)methyl]quinazoline mono-D-glucuronate.

Cipro (ciprofloxacin HCl) is commercially available from Bayer and is the monohydrochloride monohydrate salt of 1-cyclopropyl-6-fluoro-1,4-dihydro-4-oxo-7-(1-piperazinyl)-3-quinolinecarboxylic acid.

Floxin (ofloxacin) is commercially available from Ortho-McNeil Pharmaceutical and is (±)-9-fluoro-2,3-dihydro-3-methyl-10-(4-methyl-1-piperazinyl)-7-oxo-7H-pyrido[1,3,3-de]-1,4-benzoxazine-6-carboxylic acid.

Levaquin (levofloxacin) is commercially available from Ortho-McNeil Pharmaceutical) and is (−)-(S)-9-fluoro-2,3-dihydro-3-methyl-10-(4-methyl-1-piperazinyl)-7-oxo-7H-pyrido[1,2,3-de]-1,4-benzoxazine-6-carboxylic acid hemihydrate.

Mazaquin (lomefloxacin HCl) is commercially available from Unimed and is monohydrochloride salt of (±)-1-ethyl-6,8-difluoro-1,4-dihydro-7-(3-methyl-1-piperazinyl)-4-oxo-3-quinolinecarboxylic acid.

Noroxin (norfloxacin) is commercially available from Merck and is 1-ethyl-6-fluoro-1,4-dihydro-4-oxo-7-(1-piperazinyl)-3-quinolinecarboxylic acid.

Penetrex (enoxacin) is commercially available from Rhône-Poulenc Rorer and is 1-ethyl-6-fluoro-1,4-dihydro-4-oxo-7-(1-piperazinyl)-1,8-naphthyridine-3-carboxylic acid sesquihydrate.

Raxar (grepafloxacin HCl) is commercially available from Glaxo Wellcome and is (±)-1-cyclopropyl-6-fluoro-1,4-dihydro-5-methyl-7-(3-methyl-1-piperazinyl)-4-oxo-3-quinolinecarboxylic acid monochloride sesquihydrate.

Trovan (trovafloxacin mesylate) is commercially available from Pfizer and is (1α,5α,6α)-7-(6-amino-3-azabicyclo[3.1.0]hex-3-yl)-1-(2,4-difluorophenyl)-6-fluoro-1,4-dihydro-4-oxo-1,8-naphthyridine-3-carboxylic acid, monomethanesulfonate.

Zagam (sparfloxacin) is commercially available from Rh6ne-Poulenc Rorer and is 5-Amino-1-cyclopropyl-7-cis-3,5-dimethyl-1-piperazinyl)-6,8-difluoro-1,4,dihydro-4-oxo-3-quinolinecarboxylic acid.

Bactrim (trimethoprim and sulfamethoxazole) is commercially available from Roche Labs and is 2,4-diamino-5-(3,4,5-trimethoxybenzyl) pyrimidine (trimethoprim) and N¹,-(5-methyl-3-isoxazolyl)sulfanilamide (sulfamethoxazole).

Bactrim DS (trimethoprim and sulfamethoxazole double strength) is commercially available from Roche Labs and is 2,4-diamino-5-(3,4,5-trimethoxybenzyl) pyrimidine (trimethoprim) and N¹,-(5-methyl-3-isoxazolyl)sulfanilamide (sulfamethoxazole).

Pediazole (erythromicin ethylsuccinate and sulfisaxazole acetyl) is commercially available from Ross and is erythromicin 2′-(ethyl succinate) and N′acetyl sulfisoxazole (sulfisoxizole is N-(3,4-Dimethyl-5-isoxazolyl)-N-sulfanilyl acetamide.

Septra (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Septra DS (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Co-trimoxazole is a combined chemotherapeutic agent consisting of trimethoprim (T) and the sulphonamide sulphamethoxazole (S); their ratio is 1:5. It is bactericidal by virtue of a sequential blockade of the folic acid synthesis in micoorganisms. The antimicrobial spectrum of co-trimoxazole includes many Gram-positive and Gram-negative aerobes, Chlamydias, nocardias, protozoas (pneumocystis carinii), etc. In addition to its use for pneumocystis, co-trimoxazole mainly has practical importance against Gram-positive aerobes (urinary tract infections), pneumococci, and haemophilus influenzae (respiratory tract infections and otitis). http://www.infomed.org/100drugs/ctrifram.html.

Bactrim I.V. Infusion (sulfamethoxazole) is commercially available from Roche Labs.

Pediazole (erythromicin ethylsuccinate and sulfisoxazole acetyl) is commercially available from Ross and is erythromicin 2′-(ethyl succinate) and N′acetyl sulfisoxazole (sulfisoxizole is N-(3,4-Dimethyl-5-isoxazolyl)-N-sulfanilyl acetamide.

Furadantin (nitrofurantoin) is commercially available from Dura and is 1-[[(5-nitro-2-furanyl)methylene]amino]-2,4-imidazolidinedione.

Macrobid (nitrofurantoin monohydrate macrocrystals) is commercially available from Procter & Gamble Pharmaceuticals and is 1-[[[5-nitro-2-furanyl]methylene]amino]-2-4-imidazolidinedione monohydrate.

Macrodantin (nitrofurantoin macrocrystals) is commercially available from Procter & Gamble Pharmaceuticals and is 1-[[[5-nitro-2-furanyl]methylene]amino]-2-4-imidazolidinedione.

Monurol Sachet (fosfomycin tromethamine) is commercially available from Forest and is (1R,2S)-(1,2-epoxypropyl) phosphonic acid, compound with 2-amino-2-(hydroxymethyl)-1,3-propanediol (1:1).

NegGram Caplets (nalidixic acid) is commercially available from Sanofi and is 1-ethyl-1,4-dihydro-7-methyl-4-oxo-1,8-naphthyridine-3-carboxylic acid.

Septra (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Septra DS (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Urised (a combination of the antiseptics methenamine, methylene blue, phenyl salicylate, benzoic acid and parasympatholytics (atropine sulfate) hyoscyamine) is commercially available from Poly Medica.

Urobiotic-250 Capsules (oxytetracycline HCl, sulfamethizole and phenazopyridine HCl) is commercially available from Pfizer.

Uroqid Acid No. 2 Tablets (methenamine mandelate) is commercially available from Beach.

Bactroban (mupirocin) is commercially available from SmithKline Beecham and is (αE,2S,3R,4R,5S)-5-[(2S,3S,4S,5S)-2,3-Epoxy-5-hydroxy-4-methylhexyl]tetrahydro-3,4-dihydroxy-β-methyl-2H-pyran-2-crotonic acid, ester with 9-hydroxynonanoic acid, calcium salt (2:1), dihydrate.

Chloromycetin opthalmic (chloramphenical) is commercially available from Monarch and is (1) Acetamide, 2,2-dichloro-N-[2-hydroxy-1-(hydroxymethyl)-2-(4-nitrophenyl)ethyl]-, and (2) D-threo-(−)-2,2-Dichloro-N-[β-hydroxy-α-(hydroxymethyl)-p-nitrophenethyl]acetamide.

Cortisporin (neomycin and polymyxin β sulfates and hydrocortisone acetate cream) is commercially available from Monarch and is 21-(acetyloxy)-11β,17-dihydroxypregn-4-ene-3,20-dione.

Ilotycin (erythromycin opthalmic ointment) is commercially available from Dista and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-g-D-xylo-hex-opyranosyl]oxy]oxacyclotetradecane-2,10-dione.

NeoDecadron (neomycin sulfate—dexamethasone sodium phosphate) is commercially available from Merck and is 9-fluoro-11β,17-dihydroxy-16α-methyl-21-(phosphonooxy)pregna-1,4-diene-3,20-dione disodium salt.

Polytrim (trimethoprim and polythyxin β sulfate opthalmic solution) is commercially available from Allergan and is 2,4-diamino-5-(3,4,5-trimethoxylbenzl)pyrimidine(trimethoprim) and the sulfate salt of polymyxin B₁ and B₂ (polythyxin β sulfate).

Terra-Cortril (oxytetracycline HCl and hydrocortisone acetate) is commercially available from Pfizer.

TobraDex (tobramycin and dexamethasone opthalmic suspension and ointment) is commercially available from Alcon and is O-3-Amino-3-deoxy-a-D-glucopyranosyl-(1→4)-O-[2,6-diamino-2,3,6-trideoxy-a-D-ribo-hexopyranosyl-1(1→6)]-2-deoxy-L-streptamine. Dexamethasone: Chemical Name: 9-Fluro-11b,17,21-trihydroxy-16a-methylpregna-1,4-diene-3,20-dione.

Vira-A opthalmic ointment, 3% (vidarabine) is commercially available from Monarch and is 9H-Purin-6-amine, 9-β-D-arabinofuranosyl-, monohydrate.

Chibroxin (norfloxacin opthalmic solution) is commercially available from Merck and is 1-ethyl-6-fluoro-1,4-dihydro-4-oxo-7-(1-piperazinyl)-3-quinolinecarboxylic acid.

Ciloxan opthalmic solution, (Ciprofloxacin HCl) is commercially available from Alcon and is the monohydro chloride monohydrate salt of 1-cyclopropyl-6-fluoro-1,4-dihydro-4-oxo-7-(1-piperazinyl)-3-quinoline-carboxylic acid.

Ciloxan opthalmic ointment, (Ciprofloxacin HCl) is commercially available from Alcon and is the monohydro chloride monohydrate salt of 1-cyclopropyl-6-fluoro-1,4-dihydro-4-oxo-7-(1-piperazinyl)-3-quinoline-carboxylic acid.

Ocuflox opthalmic solution (ofloxacin) is commercially available from Allergan and is (±)-9-Fluoro-2,3-dihydro-3-methyl-10-(4-methyl-1-piperazinyl)-7-oxo-7H-pyrido[1,2,3-de]-1,4benzoxazine-6-carboxylic acid.

Blephamide opthalmic ointment (sulfacetamide sodium and prednisolone acetate) is commercially available from Allergan and is N-sulfanilyl-acetamide monosodium salt monohydrate (sulfacetamide sodium) and 11β,17,21-trihydroxypreyna-1,4-diene-3,20-dione 21-acetate (prednisolone acetate).

Blephamide opthalmic suspension (sulfacetamide sodium and prednisolone acetate) is commercially available from Allergan and is N-sulfanilyl-acetamide monosodium salt monohydrate ( sulfacetamide sodium) and 11β,17,21-trihydroxypreyna-1,4-diene-3,20-dione 21-acetate (prednisolone acetate).

A/T/S (erythromycin) is commercially available from Hoescht Marion Roussel and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-g-D-xylo-hex-opyranosyl]oxy]oxacyclotetradecane-2,10-dione.

Bactroban (mupirocin) is commercially available from SKB and is (αE,2S,3R,4R,5S)-5-[(2S,3S,4S,5S)-2,3-Epoxy-5-hydroxy-4-methylhexyl]tetrahydro-3,4-dihydroxy-β-methyl-2H-pyran-2-crotonic acid, ester with 9-hydroxynonanoic acid, calcium salt (2:1), dihydrate.

Benzamycin (erythromycin-benzoyl peroxide topical gel) is commercially available from Dermik and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-fl-D-xylo-hex-opyranosyl]oxy]oxacyclotetradecane-2,10-dione (erythromycin).

Betadine (povidone-iodine) is commercially available from Purdue Frederick.

Cleocin T (clindamycin phosphate topical solution) is commercially available from Pharmacia & Upjohn and is L-threo-I-D-galacto-Octopyranoside, methyl 7-chloro-6,7,8-trideoxy-6-[[(1-methyl-4-propyl-2-pyrrolidinyl)-carbonyl]amino]-1-thio-, 2-(dihydrogen phosphate), (2S-trans)-.

Clindets (clindamycin phosphate pledgets) is commercially available from Stiefel and is methyl 7-chloro-6,7,8-trideoxy-6-(1-methyl-trans-4-propyl-L-2-pyrrolidinecarboxamido)-1-thio-L-threo-α-D-galacto-octopyranoside 2-(dihydrogen phospate).

Emgel (erythromycin) is commercially available from Glaxo Wellcome and is (3R*,4S*,5S*,6R*,7R*,9R*,11R*,12R*,13S*,14R*)-4-[(2,6-Dideoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-trideoxy-3-(dimethyl-amino)-β-D-xylo-hexopyranosyl]oxy]oxacyclotetradecane-2,10-dione.

Erycette (erythromycin topical solution) is commercially available from Ortho Dermatological and is (3R*,4S*,5S*,6R*,7R*,9R*,11R*,12R*,13S*,14R*)-4-[(2,6-Dideoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-trideoxy-3-(dimethyl-amino)-β-D-xylo-hexopyranosyl]oxy]oxacyclotetradecane-2,10-dione.

Klaron (sodium sulfacetamide lotion) is commercially available from Dermik.

Mycostatin (nystatin cream) is commercially available from Westwood-Squibb.

Theramycin Z (erythromycin topical solution) is commercially available from Medicis and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-β-D-xylo-hex-opyranosyl]oxy]oxacyclotetradecane-2,10-dione.

T-Stat (erythromycin) is commercially available from Westwood-Squibb and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-β-D-xylo-hex-opyranosyl]oxy]oxacyclotetradecane-2,10-dione.

Exelderm (sulconazole nitrate) is commercially available from Westwood-Squibb and is (±)-1-[2,4-dichloro-β-[(p-chlorobenzyl)-thio]-plenethyl]imidazole mononitrate;

Fungizone (amphotericin B oral suspension) is commercially available from Bristol-Myers Squibb and is [1R-(1R*,3S*,5R*,6R*,9R*,11R*, 15S*,16R*,17R*,18S*,19E,21E,23E,25E,27E,29E,31E,33R*,35S*,36R*,37S*)]-33-[(3-Amino-3,6-dideoxy-β-D-mannopyranosyl)-oxy]-1,3,5,6,9,11,17,37-octahydroxy-15,16,18-trimethyl-13-oxo-14,39-dioxabicyclo[33.3.1]nonatriaconta-19,21,23,25,27,29,31-heptaene-36-carboxylic acid.

Lamisil (terbinafine hydrochloride cream) is commercially available from Novartis and is (E)-N-(6,6-dimethyl-2-hepten-4-ynyl)-N-methyl-1-naphthalenemethanamine hydrochloride.

Loprox (ciclopiroxolamine) is commercially available from Hoescht Marion Roussel and is 6-cyclohexyl-1-hydroxy-4-methyl-2(1H)-pyridone, 2-amino-ethanol salt.

Lotrimin (clotrimazole) is commercially available from Schering and is 1-(O-Chloro-α,α-diphenyl benzyl)imidazole.

Lotrisone (clotrimazole and betamethasone diproprionate) is commercially available from Schering and is 1-(O-Chloro-α,α-diphenyl benzyl)imidazole (clotrimazole) and 9-Fluoro-11β,17,21-trihroxy-16β-methylpregna-1,4-diene-3,20-dione 17,21-diproprionate (betamethasone diproprionate).

Mentax (butenafine HCl) is commercially available from Penederm and is N-4-tert-butylbenzyl-N-methyl-1-naphthalenemethylamine hydrochloride.

Monistat-Derm (miconazole nitrate) is commercially available from Ortho Dermatological and is 1-[2,4-dichloro-β-{(2,4-dichlorobenzyl)oxy)}phenethyl]imidazole mononitrate.

Mycelex (clotrimazole) is commercially available from Alza and is [1-(o-chloro-α,α-diphenylbenzyl)imidazole.

Mycostatin (nystatin) is commercially available from Westwood-Squibb.

Naftin (naftifine HCl) is commercially available from Allergan and is (E)-N-Cinnamyl-N-methyl-1-naphthalene-methylamine hydrochloride.

Nizoral (ketoconazole) is commercially available from Janssen and is cis-1-acetyl-4[4-[[2-(2,4-dichorophenyl)-2-(1H-imidazol-1-ylmethyl)-1,3-dioxolan-4-yl]methoxy]phenyl]piperazine.

Nystop (nystatin) is commercially available from Paddock.

Oxistat (oxiconazole nitrate) is commercially available from Glaxo Wellcome and is 2′,4′-dichloro-2-imidazole-1-ylacetophenone (Z)-[O-(2,4-dichlorobenzyl)oxime], mononitrate.

Selsun Rx (2.5% selenium sulfide lotion) is commercially available from Ross.

Spectazole (econazole nitrate) is commercially available from Ortho Dermatological and is 1-[2-{(4-chorophenyl)methoxy}-2-(2,4-dichlorophenyl)-ethyl]-1H-imidazole mononitrate.

Denavir (penciclovir cream) is commercially available from SmithKline Beecham and is 9-[4-hydroxy-3-(hydroxymethyl)butyl]guanine.

Zovirax (acyclovir) is commercially available from Glaxo-Wellcome and is 2-amino-1,9-dihydro-9-(2-hydroxyethoxy)methyl-6H-purin-6-one.

Benzashave (benzoyl peroxide) is commercially available from Medicis.

Betadine (povidone-iodine) is commercially available from Purdue Frederick.

Betasept (chlorhexidine gluconate) is commercially available from Purdue Frederick.

Cetaphil (soap substitute) is commercially available from Galaderma.

Clorpactin WCS-90 (sodium oxychlorosene) is commercially available from Guardiam Laboratories.

Dapsone Tablets (dapsone) is commercially available from Jacobus and is 4,4′-diaminodiphenyl sulfone (DDS).

Desquam-E (benzoyl peroxide) is commercially available from Westwood-Squibb.

Desquam-X (benzoyl peroxide) is commercially available from Westwood-Squibb.

Hibiclens (chlorhexidine gluconate) is commercially available from Zeneca.

Hibistat (chlorhexidine gluconate) is commercially available from Zeneca.

Impregon (tetrachlorosalicylanilide 2%) is commercially available from Fleming.

MetroCream (metronidazole) is commercially available from Galaderma and is 2-methyl-5-nitro-1H-imidazole-1-ethanol.

MetroGel (metronidazole) is commercially available from Galaderma and is 2-methyl-5-nitro-1H-imidazole-1-ethanol.

Noritate (metronidazole) is commercially available from Dermik and is 2-methyl-5-nitro-1H-imidazole-1-ethanol.

pHisoHex (hexachlorophene detergent cleanser) is commercially available from Sanofi and is Phenol,2,2′-methylene-bis[3,4,6-trichloro-].

Sulfacet-R (sodium sulfacetamide 10% and sulfur 5%) is commercially available from Dermik.

Sulfamylon (matenide acetate) is commercially available from Bertek and is α-amino-p-toluenesulfonamide monoacetate.

Triaz (benzoyl peroxide) is commercially available from Medicis.

Vanoxide-HC (benzoyl peroxide hydrocortisone) is commercially available from Dermik and is 11β,17,21-trihydroxypregn-4-ene-3,20-dione (hydrocortisone).

Acticin (permethrin) is commercially available from Penederm and is (±)-3-phenoxybenzyl 3-(2,2-dichlorovinyl)-2,2-dimethylcyclopropanecarboxylate.

Elimite (permethrin) is commercially available from Allergan and is (±)-3-phenoxybenzyl 3-(2,2-dichlorovinyl)-2,2-dimethylcyclopropanecarboxylate.

Eurax (crotamiton) is commercially available from Westwood-Squibb and is N-ethyl-N-(o-methylphenyl)-2-butenamide.

Lindane Lotion USP 1% (lindane) is commercially available from Alpharma.

Efudex (fluorouracil) is commercially available from ICN and is 5-flouro-2,4(1H,3H)-pyrimidinedione.

Fluoroplex (fluorouracil) is commercially available from Allergan and is 5-flouro-2,4(1H,3H)-pyrimidinedione.

Furadantin Oral Suspension (nitrofurantoin) is commercially available from Dura and is 1-[[5-nitro-2-furanyl)methylene]amino]-2,4-imidazolidine dione.

Zyvox (linezolid) is commercially available from Pharmacia & Upjohn.

It is appreciated that those skilled in the art understand that the antibiotic useful in the present invention is the biologically active compound present in any of the antibiotic drugs disclosed above. For example, Azactam (aztreonam) is typically available as an injectable solution. The antibiotic, however, is (z)-2-[[[(2-amino-4-thiazolyl)[[(2S,-3S)-2-methyl-4-oxo-1-sulfo-3-azetidinyl]carbamoyl]methylene]amino]oxy]-2-methyl propionic acid. Physician's Desk Reference (PDR), Medical Economics Company (Montvale, N.J.), (53rd Ed.), pp. 820-823. 1999.

Anti-Inflammatory Steroid

Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. In some embodiments, the steroidal anti-inflammatory used is hydrocortisone.

Non-Steroidal Anti-Inflammatory Drug (NSAID)

A second class of anti-inflammatory agents that is useful in the compositions includes the nonsteroidal anti-inflammatory agents. A variety of compounds are encompassed by this group. For detailed disclosure of the chemical structure, synthesis, side effects, etc. of non-steroidal anti-inflammatory agents, one may refer to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974).

Specific non-steroidal anti-inflammatory agents useful in the composition invention include, but are not limited to:
1) the oxicams, such as piroxicam, isoxicam, tenoxicam, sudoxicam, and CP-14,304;
2) benorylate, trilisate, safapryn, solprin, diflunisal, and fendosal;
3) the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;
4) the fenamates, such as mefenamic, meclofenamic, flufenamic, niflumic, and tolfenamic acids;
5) the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and
6) the pyrazoles, such as phenylbutazone, oxyphenbutazone, feprazone, azapropazone, and trimethazone.

Mixtures of these non-steroidal anti-inflammatory agents may also be employed, as well as the dermatologically acceptable salts and esters of these agents. For example, etofenamate, a flufenamic acid derivative, is useful for topical application. Of the nonsteroidal anti-inflammatory agents, ibuprofen, naproxen, flufenamic acid, etofenamate, aspirin, mefenamic acid, meclofenamic acid, piroxicam and felbinac are often used. Ibuprofen, naproxen, ketoprofen, etofenamate, aspirin and flufenamic acid are frequently used.

Moreover, so-called “natural” anti-inflammatory agents are useful in methods of the present invention. Such agents may suitably be obtained as an extract by suitable physical and/or chemical isolation from natural sources (e.g., plants, fungi, by-products of microorganisms) or can be synthetically prepared. For example, candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commiphora, particularly Commiphora Mukul), kola extract, chamomile, red clover extract, and sea whip extract, may be used.

Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, or C₁₀-C₂₄, or C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred.

Specifically, the non-steroidal anti-inflammatory (NSAID) drug can selected from the group of aspirin, meclofenamate sodium, oxyphenbutazone, phenylbutazone, indomethacin, piroxicam, sulindac and tolmetin; mefenamic acid and zomepirac; ibuprofen, fenoprofen and naproxen.

Glutathione Enhancing Agent

Glutathione enhancing agents can be employed in the compositions and methods of the present invention to increase the intracellular levels of GSH in patients suffering from different stages and forms of macular degeneration. Specifically, the intracellular levels of GSH can be increased by periodically administering to the patient a dosage of a cysteine-supplying derivative, preferably N-acetylcysteine (NAC) or its equivalent. NAC is a known mucolytic agent. It is the N-acetyl derivative (HSCH₂CHCOOHNHCOCH₃) of the naturally occurring amino acid, L-cysteine. NAC is more soluble than cysteine in water and is less easily oxidized than cysteine. Hence, it is preferred to use NAC or an equivalent cysteine derivative rather than cysteine itself. It is believed, however, that once administered, it is cysteine, which functions to increase GHS levels, and consequently, to treat or prevent tissue damage such as that observed in macular degeneration.

It is not fully understood how cysteine or its derivatives function to accomplish the intended treatment. However, it appears that the possible protective mechanisms offered by N-acetylcysteine (NAC) include: scavenging (inactivation of) oxygen radicals (e.g. H₂O₂, HOCl or ⁻OH) directly; inhibiting NFkB nuclear factor activation, which is a known link between viral infection and activation of oxidative processes; decreasing oxidant-induced lipid peroxidation and oxidant-induced inactivation of anti-proteases; and increasing blood flow, perhaps as a consequence of a potentiating action with nitric oxide.

NAC is a particularly desirable compound for use according to the invention as it is currently used clinically to treat patients with, for example, chronic bronchitis, acetaminophen overdose, and mustard gas exposure. NAC is also being tested as a treatment for ARDS, AIDS, ALS, and other conditions. NAC has been used safely for many years. For example, an oral dose of 600 mg tid is well tolerated for long periods of time in patients with chronic bronchitis and emphysema. Radiolabelled NAC is rapidly absorbed in humans one hour after administration and then distributed extensively. The mean plasma half-life of NAC is about 1.35 hours and approximately 22% of the dose is excreted in the urine after 24 hours. NAC appears to bind to protein and undergo some metabolism. Adverse effects following NAC treatment are rare but can include nausea, vomiting, pyrosis, dyspepsia, and very rarely, urticaria. These possible side effects can, however, generally be moderated to enable the treatment contemplated herein.

NAC is also advantageous for the present treatment of macular degeneration because it appears to augment anti-oxidant defense mechanisms by enhancing glutathione redox cycle activity. Specifically, NAC enables cysteine to penetrate cells. Increasing intracellular cysteine levels increases production of intracellular GSH which facilitates glutathione redox cycle activity (GSH.revreaction.GSSG). The effect of increased glutathione redox cycle activity is the continued detoxification of hydrogen peroxide (H₂O₂) and related hydroperoxides (e.g., hypochlorous acid and hydroxyl radicals). Hydrogen peroxide and hydrogen peroxide derived products are directly toxic to key cellular molecules (e.g., lipids, DNA, and proteins), and can accelerate inflammation. By increasing intracellular GSH levels, NAC treatment may reduce the toxic levels of intracellular hydrogen peroxide and related hydroperoxides that often relate to ocular tissue damage, specifically macular degeneration.

It is believed that intracellular GSH levels decline with age, and as a consequence, decreased GSH levels may enhance oxidative stress which contributes to ocular tissue damage. With the compositions and methods described herein, NAC can be useful to prevent or reverse the ocular dysfunctions typical of elderly patients that would otherwise develop with macular degeneration.

In a variation of the invention, intracellular levels of GSH are increased by periodically administering to the patient a dosage of L-2-oxothiazolidine-4-carboxylate (OTC). OTC, or procysteine as it is commonly known, is effectively transported into cells where it is converted by the action of 5-oxoprolinase into L-cysteine. By mechanisms similar to those described above with respect to NAC, OTC can indirectly affect increased levels of GSH and thereby augment anti-oxidant defense mechanisms by enhancing glutathione redox cycle activity. OTC has been shown to be effectively transported into mouse and rat brains as evidenced by increased brain cystsine levels in the mouse and rat following OTC administration. The dosage of OTC used for present purposes, and the mode of administration are generally similar to those given above for NAC.

In another variation of the invention, intracellular levels of GSH are increased by periodically administering to the patient a dosage of mercaptopropionylglycine (MPG). MPG increases GSH levels by decreasing oxidative damage both in vivo and in vitro (i.e., MPG-reduced myocardial infract size following ischemia-reperfusion). Free radical scavenging has been postulated as a mechanism for the protective effect of MPG. When measured by pulse radiolysis in vitro, MPG has been shown to scavenge OH (hydroxyl radicals) at a rate constant of approximately 10⁹ M⁻¹s⁻¹. Experimental pretreatment with MPG has also been shown to increase free sulfhydryl (GSH) content in control animals and maintain normal free sulfhydryl levels in animals subjected to ischemia-reperfusion. Although the protective mechanism(s) by which MPG functions for present purposes is not known, there are several possible explanations. Thus, for example, GSH levels in cell tissue might be increased or maintained by MPG-induced glutathione synthesis and/or MPG-induced release of protein bound GSH. An alternative possibility is that GSH is spared by MPG. A further possibility is that MPG might act as a sulfhydryl radioprotector. In any case, the MPG appears to function as a GSH enhancing agent useful for present purposes.

The amount of MPG which is used and the mode of administration can also be varied, as in the case of NAC and OTC.

A unique aspect of the invention utilizing NAC, OTC, or MPG alone, or in a combination of two or more, or in conjunction with one or more anti-oxidant, anti-inflammatory, or current symptomatic treatments, is that direct treatment with GSH or cysteine would not be beneficial. An apparent reason for this is that neither unacetylated cysteine, nor GSH appear to penetrate cells by themselves. Moreover, cysteine itself may be toxic if it is oxidized extracellularly. Likewise, other anti-oxidants, for example antioxidant vitamins such as vitamin A, lutein, zeaxanthin, vitamin E, beta-carotene, or vitamin C, may not be as effective because they also do not penetrate cells easily.

Macular degeneration can also occur in localized areas which are not recognized by routine examination until severe and often permanent eye destruction occurs. An additional advantage of the claimed method of treatment is that this problem can be overcome with a nontoxic treatment, such as NAC, OTC, or MPG, which can be used proactively to prevent the development of forms of macular degeneration which could not otherwise be detected earlier.

The invention contemplates employing in the compositions described herein, any agent which can enhance intracellular GSH levels to treat or prevent the onset or progression of macular degeneration. Preferably, NAC, OTC and/or MPG are used for this purpose but it will be appreciated that other GSH enhancing agents may also be useful.

Ocular Neovascular Disease

Ocular neovascular disease is characterized by invasion of new blood vessels into the structures of the eye such as the retina or cornea. It is the most common cause of blindness and is involved in approximately twenty eye diseases. In advanced age-related macular degeneration, the associated visual problems are caused by an ingrowth of chorioidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical bums, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, and comeal graph rejection.

Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy.

Macular Degeneration

A safe and effective method of preventing, stabilizing, reversing and/or treating macular degeneration or visual acuity loss by reducing the risk of developing late stage or advanced age-related macular degeneration in persons with early age-related macular degeneration and/or by reducing the risk of vision loss associated with the development of cataracts are provided with the methods and compositions of the present invention.

Angiogenesis-Related Diseases

Angiogenesis-related diseases may be diagnosed and treated using the endothelial cell proliferation inhibiting compounds of the present invention. A ngiogenesis-related diseases include, but are not limited to, ocular angiogenic diseases, for example, diabetic retinopathy, retinopathy of prematurity, macular degeneration, corneal graft rejection, neovascular glaucoma, retrolental fibroplasia, rubeosis; angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; Osler-Webber Syndrome; myocardial angiogenesis; plaque neovascularization; telangiectasia; hemophiliac joints; angiofibroma; and wound granulation. The endothelial cell proliferation inhibiting proteins of the present invention are useful in the treatment of disease of excessive or abnormal stimulation of endothelial cells. These diseases include, but are not limited to, intestinal adhesions, atherosclerosis, scleroderma, and hypertrophic scars, i.e., keloids. They are also useful in the treatment of diseases that have angiogenesis as a pathologic consequence such as cat scratch disease (Rochele minalia quintosa) and ulcers (Helobacter pylori).

Dosage and Formulation

The composition can be administered as treatment, e.g., for heart disease, macular degeneration, cancer, and related diseases and symptoms, by any means that produces contact of the composition with the agent's site of action in the body of a mammal. The composition can be administered by any conventional means available for use in conjunction with pharmaceuticals or neutraceuticals. The composition can be administered alone, but preferably is administered with a pharmaceutical or neutraceutical carrier selected on the basis of the chosen route of administration and standard pharmaceutical or neutraceutical practice.

Specifically, the compositions of the present invention can be administered systemically or locally. In one embodiment of the present invention, the compositions of the invention can be administered locally through a variety of delivery systems including eye drops. In another embodiment of the present invention, the compositions of the invention can be administered systemically through a variety of delivery systems including orally, liposomes, parenteral injection (e.g., intravenously, intramuscularly or subcutaneously), transderrnally, aerosolization (e.g., inhaler), or direct injection (e.g., intravitreal).

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of composition can be expected to be about 0.001 to about 1 gram per kilogram of body weight, with the preferred daily dose being split and administered over several times a day.

The dosage can be administered one or more times per day (e.g., once per day, twice per day, three times per day, or more).

The composition can be administered orally in solid dosage forms, such as chewable lozenges, capsules, tablets and powders, or in liquid dosage forms, such as elixirs, syrups and suspensions. It can also be administered parenterally, in sterile liquid dosage forms. Additives may also be included in the formulation to enhance the physical appearance, improve stability, and aid in disintegration after administration. For example, liquid dosage forms for oral administration can contain coloring and flavoring to increase patient acceptance.

Gelatin capsules contain the composition and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours or days. Sustained release products can also be formulated for implantation or transdermal/transmucosal delivery. Such formulations typically will include a polymer that biodegrades or bioerodes thereby releasing a portion of the composition. The formulations may have the form of microcapsules, liposomes, solid monolithic implants, gels, viscous fluids, discs, or adherent films.

Compressed tablets can be sugar coated or film coated to mask any unpleasant taste and protect the tablet from the atmosphere, or enteric coated for selective disintegration in the gastrointestinal tract.

Film-coated tablets are compressed tablets, which are covered with as thin layer of film or water-soluble material. A number of polymeric substances with film-forming properties may be used. Film coating imparts the same general characteristics as sugar coating with the added advantage of a greatly reduced time period required for the coating operation.

Enteric-coated tablets are compressed tablets coated with substances that resist solution in gastric fluid but disintegrate in the intestine. Enteric coatings can be used for tablets containing drug substances which are inactivated or destroyed in the stomach, for those which irritate the mucosa, or as a means of delayed release of the medication.

Multiple compressed tablets are compressed tablets made by more than one compression cycle.

Layered tablets are prepared by compressing additional tablet granulation on a previously compressed granulation. The operation my be repeated to produce multilayered tablets of two or three layers. Special tablet presses are required to make layered tablets.

Press-coated tablets, which are also referred to as dry-coated, are prepared by feeding previously compressed tablets into a special tableting machine and compressing another granulation layer around the preformed tablets. They have all the advantages of compressed tablets, i.e., slotting, monogramming, speed of disintegration, etc., while retaining the attributes of sugar-coated tablets in masking the taste of the drug substance in the core tablets. Press-coated tablets can also be used to separate incompatible drug substances; in addition, they can provide a means to give an enteric coating to the core tablets. Both types of multiple-compressed tablets have been widely used in the design of prolonged-action dosage forms.

Compressed tablets can be formulated to release the drug substance in a manner to provide medication over a period of time. There are a number of types which include delayed-action tablets in which the release of the drug substance is prevented for an interval of time after administration of until certain physiological conditions exist; repeat-action tablets which periodically release a complete dose of the drug substance to the gastrointestinal fluids; and the extended-release tablets which continuously release increments of the contained drug substance to the gastrointestinal fluids.

The non-aqueous carrier, or excipient, can be any substance that is biocompatible and liquid or soft enough at the mammal's body temperature to release the composition into the animal's bloodstream at a desired rate. The carrier is usually hydrophobic and commonly organic, e.g., an oil or fat of vegetable, animal, mineral or synthetic origin or derivation. Preferably, but not necessarily, the carrier includes at least one chemical moiety of the kind that typifies “fatty” compounds, e.g., fatty acids, alcohols, esters, etc., i.e., a hydrocarbon chain, an ester linkage, or both. “Fatty” acids in this context include acetic, propionic and butyric acids through straight- or branched-chain organic acids containing up to 30 or more carbon atoms. Preferably, the carrier is immiscible in water and/or soluble in the substances commonly known as fat solvents. The carrier can correspond to a reaction product of such a “fatty” compound or compounds with a hydroxy compound, e.g., a mono-hydric, di-hydric, trihydric or other polyhydric alcohol, e.g., glycerol, propanediol, lauryl alcohol, polyethylene or -propylene glycol, etc. These compounds include the fat-soluble vitamins, e.g., tocopherols and their esters, e.g., acetates sometimes produced to stabilize tocopherols. Sometimes, for economic reasons, the carrier may preferably comprise a natural, unmodified vegetable oil such as sesame oil, soybean oil, peanut oil, palm oil, or an unmodified fat. Alternatively the vegetable oil or fat may be modified by hydrogenation or other chemical means which is compatible with the present invention. The appropriate use of hydrophobic substances prepared by synthetic means is also envisioned.

Typically, water, suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the composition, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts, and sodium EDTA. In addition, parenteral solutions can contain preservatives, such as benzalkonium chloride, methyl- or propyl-paraben and chlorobutanol. Suitable pharmaceutical carriers are described in Remington's Pharmaceutical Sciences, supra, a standard reference text in this field.

In addition to the active or therapeutic ingredient, tablets contain a number of inert materials. The latter are known as additives or “adds.” They may be classified according to the part they play in the finished tablet. The first group contains those which help to impart satisfactory compression characteristics to the formulation. These include (1) diluents, (2) binders, and (3) lubricants. The second group of added substances helps to give additional desirable physical characteristics to the finished tablet. Included in this group are (1) disintegrators, (2) colors, and in the case of chewable tablets, (3) flavors, and (4) sweetening agents.

Frequently the single dose of the composition is small and an inert substance is added increase the bulk in order to make the tablet a practical size for compression. Diluents used for this purpose include dicalcium phosphate, calcium sulfate, lactose, kaolin, mannitol, sodium chloride, dry starch, and powdered sugar.

Most tablet formulators tend to use consistently only one or two diluents selected from the above group in their tablet formulations. Usually these have been selected on the basis of experience and cost factors. However, the compatibility of the diluent with the drug must be considered. When drug substances have low water solubility, it is recommended that water-soluble diluents be used to avoid possible bioavailability problems.

Agents used to impart cohesive qualities to the powdered material are referred to as binders or granulators. They impart a cohesiveness to the tablet formulation which insures the tablet remaining intact after compression, as well as improving the free-flowing qualities by the formulation of granules of desired hardness and size. Materials commonly used as binders include starch, gelatin, and sugars as sucrose, glucose, dextrose, molasses, and lactose. Natural and synthetic gums which have been used include acacia, sodium alginate, extract of Irish moss, panwar gum, ghatti gum, mucilage of isapol husks, carboxymethylcellulose, methylcellulose, polyvinylpyrrolidone, Beegum, and larch arabogalactan. Other agents which may be considered binders under certain circumstances are polyethylene glycol, ethylcellulose, waxes, water and alcohol.

The quality of binder used has considerable influence on the characteristics of the compressed tablets. The use of too much binder or too strong a binder will make a hard tablet which will not disintegrate easily. Alcohol and water are not binders in the true sense of the word; but because of their solvent action on some ingredients such as lactose and starch, they change the powdered material to granules and the residual moisture retained enables the materials to adhere together when compressed.

Lubricants have a number of functions in tablet manufacture. They improve the rate of flow of the tablet granulation, prevent adhesion of the tablet material to the surface of the dies and punches, reduce interparticle friction, and facilitate the ejection of the tablets from the die cavity. Commonly used lubricants include talc, magnesium stearate, calcium stearate, stearic acid, and hydrogenated vegetable oils. Most lubricants with the exception of talc are used in concentrations less than 1%. Lubricants are in most cases hydrophobic materials. Poor selection or excessive amounts can result in “waterproofing” the tablets, result in poor tablet disintegration and dissolution of the drug substance.

A disintegrator is a substance, or a mixture of substances, added to a tablet to facilitate its breakup or disintegration after administration. The composition must be released from the tablet matrix as efficiently as possible to allow for its rapid dissolution. Materials serving as disintegrates have been chemically classified as starches, clays, celluloses, aligns, or gums.

The most popular disintegrators are corn and potato starch which have been well-dried and powdered. Starch has a great affinity for water and swells when moistened, thus facilitating the rupture of the tablet matrix. However, others have suggested that its disintegrating action in tablets is due to capillary action rather than swelling; the spherical shape of the starch grains increases the porosity of the tablet, thus promoting capillary action.

In addition to the starches a large variety of materials have been used and are reported to be effective as disintegrators. This group includes Veegum HV, methylcellulose, agar, bentonite, cellulose and wood products, natural sponge, cation-exchange resins, alginic acid, guar gum, citrus pulp, and carboxymethylcellulose. Sodium lauryl sulfate in combination with starch also has been demonstrated to be an effective disintegrant.

Colors in compressed tablets serve functions other than making the dosage from more esthetic in appearance. Any of the approved certified water-soluble FD&C dyes, mixtures of the same, or their corresponding lakes may be used to color tablets.

In addition to the sweetness which may be afforded by the diluent of the chewable tablet, e.g. mannitol or lactose, artificial sweetening agents may be included. Among the most promising are two derivatives of glycyrrhizin, the glycoside obtained from licorice.

Compressed tablets may be characterized or described by a number of specifications. These include the diameter size, shape, thickness, weight, hardness, and disintegration time.

Useful pharmaceutical, neutraceutical, and/or cosmeceutical dosage-forms for administration of the composition can be illustrated as follows:

Capsules

A large number of unit capsules can be prepared by filling standard two-piece hard gelatin capsules each with 100 mg of powdered composition, 150 mg of lactose, 50 mg of cellulose, and 6 mg magnesium stearic.

Soft Gelatin Capsules

A mixture of composition in digestible oil such as soybean oil, cottonseed oil or olive oil can be prepared and injected by means of a positive displacement pump into gelatin to form soft gelatin capsules containing 100 mg of the composition. The capsules should then be washed and dried.

Tablets

A large number of tablets can be prepared by conventional procedures so that the dosage unit is 100 mg of composition, 0.2 mg of colloidal silicon dioxide, 5 milligrams of magnesium stearate, 275 mg of microcrystalline cellulose, 11 mg of starch and 98.8 mg of lactose.

Suspension

An aqueous suspension can be prepared for oral administration so that each 5 mL contain 25 mg of finely divided composition, 200 mg of sodium carboxymethyl cellulose, 5 mg of sodium benzoate, 1.0 g of sorbitol solution, U.S.P., and 0.025 mg of vanillin.

Sport Beverage

An aqueous fluid can be prepared for oral administration so that each 355 mL contains 200 mg of finely divided composition, 250 mg of fructose or sucrose, 5 mg of sodium benzoate, 1 mg of colorant, 5 mg of flavoring agent, and 25 mg of potassium chloride in water (350 ml). Alternatively, the finely divided composition (200 mg) can be present in orange juice (355 ml).

Injectable

A parenteral composition suitable for administration by injection can be prepared by stirring 1.5% by weight of composition in 10% by volume propylene glycol and water. The solution is sterilized by commonly used techniques.

Pediatric Dosage

For children, a daily dosage of composition can be expected to be about 0.001 milligrams per kilogram of body weight to about 100 milligrams per kilogram of body weight, with the preferred dose being about 0.1 mg/kg to about 50 mg/kg, preferably administered several times a day. More specifically, the preferred dose be about 1 mg/kg to about 25 mg/kg, preferably administered several times a day.

Dosage forms of compositions suitable for administration to children will contain from about 1 mg to about 50 mg of composition per unit. Specifically, the dosage form can contain from about 5 mg to about 25 mg of composition per unit.

Eye Drop

A composition suitable for ocular administration can be prepared by stirring 1.5% by weight of composition in 10% by volume saline and buffer. The solution is sterilized by commonly used techniques.

### Enumerated Embodiments of the Invention

[1.]The present invention provides a composition comprising: (a) xanthophylls; (b) vitamin C; (c) vitamin E; (d) zinc; and (e) copper.
[2.]The present invention provides the composition of embodiment [1], wherein the xanthophylls comprise lutein, zeaxanthin, capsorubin, capsanthin, astaxanthin, canthaxanthin, or any combination thereof.
[3.]The present invention provides the composition of embodiment [1], wherein the xanthophylls comprise lutein and zeaxanthin.
[4.]The present invention provides the composition of embodiment [1], wherein the xanthophylls comprise lutein and zeaxanthin, and wherein the lutein is provided in the non-esterified form.
[5.]The present invention provides the composition of embodiment [1], wherein the xanthophylls comprise lutein and zeaxanthin, and wherein the lutein is provided as trans-lutein.
[6.]The present invention provides the composition of embodiment [1], wherein the xanthophylls comprise lutein and zeaxanthin, and wherein the lutein is provided as trans-lutein that is at least about 50 wt. % pure.
[7.]The present invention provides the composition of embodiment [1], wherein the xanthophylls comprise lutein and zeaxanthin, and wherein the lutein is provided as trans-lutein that is about 50 wt. % to about 90 wt. % pure.
[8.]The present invention provides the composition of any one of embodiments [1]-[7], wherein the vitamin C is provided in the form of ascorbic acid.
[9.]The present invention provides the composition of any one of embodiments [1]-[8], wherein the vitamin E is provided in the form of dl-alpha tocopheryl acetate.
[10.]The present invention provides the composition of any one of embodiments [1]-[9], wherein the zinc is provided in the form of zinc oxide, zinc gluconate or a combination thereof.
[11.]The present invention provides the composition of any one of embodiments [1]-[10], wherein the copper is provided in the form of cupric oxide, copper gluconate or a combination thereof.
[12.]The present invention provides the composition of any one of embodiments [1]-[11], wherein the composition is provided as a daily dosage.
[13.]The present invention provides the composition of any one of embodiments [1]-[12], wherein the composition is provided as a daily dosage, and the xanthophylls are present in about 6 times to about 10 times the Recommended Daily Allowance (RDA).
[14.]The present invention provides the composition of any one of embodiments [1]-[12], wherein the xanthophylls are present in about 10.0 mg to about 50 mg.
[15.]The present invention provides the composition of any one of embodiments [1]-[12], wherein the xanthophylls are present in about 17.0 mg to about 28 mg.
[16.]The present invention provides the composition of any one of embodiments [1]-[15], wherein the composition is provided as a daily dosage, and the vitamin C is present in about 7 times to about 10 times the Recommended Daily Allowance (RDA).
[17.]The present invention provides the composition of any one of embodiments [1]-[15], wherein the vitamin C is present in about 200 mg to about 800 mg.
[18.]The present invention provides the composition of any one of embodiments [1]-[15], wherein the vitamin C is present in about 420 mg to about 600 mg.
[19.]The present invention provides the composition of any one of embodiments [1]-[18], wherein the composition is provided as a daily dosage, and the vitamin E is present in about 13 times to about 18 times the Recommended Daily Allowance (RDA).
[20.]The present invention provides the composition of any one of embodiments [1]-[18], wherein the vitamin E is present in about 200 IU to about 700 IU.
[21.]The present invention provides the composition of any one of embodiments [1]-[18], wherein the vitamin E is present in about 400 IU to about 540 IU.
[22.]The present invention provides the composition of any one of embodiments [1]-[21], wherein the composition is provided as a daily dosage, and the zinc is present in about 4 times to about 7 times the Recommended Daily Allowance (RDA).
[23.]The present invention provides the composition of any one of embodiments [1]-[21], wherein the zinc is present in about 40 mg to about 150 mg.
[24.]The present invention provides the composition of any one of embodiments [[1]-[21], wherein the zinc is present in about 60 mg to about 100 mg.
[25.]The present invention provides the composition of any one of embodiments [1]-[24], wherein the composition is provided as a daily dosage, and the copper is present in at least about 1.6 mg.
[26.]The present invention provides the composition of any one of embodiments [1]-[24], wherein the copper is present in about 1.6 mg to about 2.4 mg.
[27.]The present invention provides the composition of any one of embodiments [1]-[26], wherein the composition is formed into one or more tablets for daily oral ingestion by a human.
[28.]The present invention provides the composition of any one of embodiments [1]-[26], wherein the composition is formed into four tablets for oral ingestion by a patient of two tablets twice daily.
[29.]The present invention provides the composition of any one of embodiments [1]-[28], wherein the composition further comprises a pharmaceutically acceptable carrier or a neutraceutically acceptable carrier.
[30.]The present invention provides the composition of any one of embodiments [1]-[29], wherein the composition is in the form of a powder, eye drop, liposome, tablet, capsule, gel, liquid or solid.
[31.]The present invention provides the composition of any one of embodiments [1]-[30], wherein the composition is admixed with a food product.
[32.]The present invention provides the composition of any one of embodiments [1]-[30], wherein the composition is admixed with a food beverage.
[33.]The present invention provides the composition of any one of embodiments [1]-[30], wherein the composition is admixed with orange juice.
[34.]The present invention provides the composition of any one of embodiments [1]-[30], wherein the composition is admixed with a sport drink or a health bar.
[35.]The present invention provides the composition of any one of embodiments [1]-[34], further comprising alpha-lipoic acid, a phenolic compound, an anthocyanoside, or a combination thereof.
[36.]The present invention provides the composition of any one of embodiments [1]-[35], further comprising beta-carotene, vitamin A, or a combination thereof.
[37.]The present invention provides the composition of any one of embodiments [1]-[36], further comprising melanin.
[38.]The present invention provides the composition of any one of embodiments [1]-[37], further comprising a melanin-promoting compound.
[39.]The present invention provides the composition of any one of embodiments [1]-[38], further comprising a phytoestrogen selected from the group of genistein, genistin, 6″-O-Mal genistin, 6″-O—Ac genistin, daidzein, daidzin, 6″-O-Mal daidzin, 6″-O—Ac daidzin, glycitein, glycitin, 6″-O-Mal glycitin, biochanin A, formononetin, or a combination thereof.
[40.]The present invention provides the composition of embodiment [39], wherein at least one of the phytoestrogens in the composition is an antioxidant.
[41.]The present invention provides the composition of embodiment [39], wherein at least one of the phytoestrogens in the composition a tyrosine kinase inhibitor.
[42.]The present invention provides the composition of embodiment [39], wherein at least one of the phytoestrogens in the composition is an angiogenesis inhibitor.
[43.]The present invention provides the composition of embodiment [39], wherein at least one of the phytoestrogens in the composition is effective to lower LDL cholesterol concentration in the blood of the human.
[44.]The present invention provides the composition of embodiment [39], wherein at least one of the phytoestrogens in the composition has estrogenic activity.
[45.]The present invention provides the composition of embodiment [39], wherein at least one of the phytoestrogens in the composition is a vasodilitory agent.
[46.]The present invention provides the composition of embodiment [39], wherein at least one of the phytoestrogens in the composition is derived from soy or clover.
[47.]The present invention provides the composition of embodiment [39], wherein the phytoestrogen is present in about 0.1 mg to about 2000 mg of the composition.
[48.]The present invention provides the composition of embodiment [39], wherein the phytoestrogen is present in about 20 mg to about 1000 mg of the composition.
[49.]The present invention provides the composition of any one of embodiments [1]-[48], further comprising an antibiotic.
[50.]The present invention provides the composition of any one of embodiments [1]-[49], further comprising an antibiotic selected from the group of an aminoglycoside, β-lactam antibiotic, cephalosporin, macrolide, miscellaneous antibiotic, penicillin, tetracycline, antifungal, antimalarial agent, antituberculosis agent, antiviral, leprostatic, miscellaneous anti-infectives, quinolone, sulfonamide, urinary anti-infective, nasal antibiotic, opthalmic antibiotic, opthalmic antiviral, opthalmic quinalone, opthalmic sulfonamide, skin and mucous membrane antibiotic, skin and mucous membrane antifungal, skin and mucous membrane antiviral, skin and mucous membrane miscellaneous anti-infective, skin and mucous membrane scabicide or pedulicide, skin and mucous membrane antinepolast, and combinations thereof.
[51.]The present invention provides the composition of any one of embodiments [1]-[50], further comprising an anti-inflammatory steroid.
[52.]The present invention provides the composition of any one of embodiments [1]-[51], further comprising an anti-inflammatory steroid selected from the group of corticosteroids, hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amncinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used.
[53.]The present invention provides the composition of any one of embodiments [1]-[52], further comprising a non-steroidal anti-inflammatory drug (NSAID).
[54.]The present invention provides the composition of any one of embodiments [1]-[53], further comprising a non-steroidal anti-inflammatory (NSAID) drug selected from the group of aspirin, meclofenamate sodium, oxyphenbutazone, phenylbutazone, indomethacin, piroxicam, sulindac and tolmetin; mefenamic acid and zomepirac; ibuprofen, fenoprofen and naproxen.
[55.]The present invention provides the composition of any one of embodiments [1]-[54], further comprising a glutathione enhancing agent.
[56.]The present invention provides the composition of any one of embodiments [1]-[55], further comprising a glutathione enhancing agent selected from the group of N-acetylcysteine, L-2-oxothiazolidine-4-carboxylate, mercaptopropionylglycine, and combinations thereof.
[57.]The present invention provides the composition of any one of embodiments [1]-[56], further comprising an angiotensin converting enzyme inhibitor selected from the group of enalapril, enalaprilat, lisinopril, captopril, ranipril, perindopril, zofenopril, quinapril, pentopril, cilazapril, pivopril, fosenopril, indolapril, indalapril, phenacein, fentiapril, alacepril, perinodopril, mugenic acid, ancovenin; 2-[2-[[1-(1-(ethoxycarbonyl)-3-phenylpropyl]amino]-1-oxopropyl-6,7-dimethoxy-1,2,3,4-tetrahydro-3-isoquinolinecarboxylic acid; 3-[[1-ethoxycarbonyl-3-phenyl-(1S) propyl]amino]-2,3,4,5-tetrahydro-2-oxo-1-(3S)benzazepine-1-acetic acid HCl; 3-[[1-carboxylate-3-phenyl-1(S)propyl]-amino]-2,3,4,5-tetrahydro-2-oxo-1(3S)benzazepine-1-acetic acid HCl; 2[2-[[1-carbonyl-3-phenylpropyl]-amino]-1-oxopropyl]-1,2,3,4-tetra-hydro-3-isoquinoline-carboxylic acid; [1-(S)4S]4-[[2-[6-(aminosulfonyl)-7-chloro-1,2,3,4-tetrahydro-4-oxo-2-quinazolinyl]ethyl]-thio]-1-(3-mercapto-2-methyl-1-oxo-propyl)-L-proline monosodium salt; [1-(s)4S]4-[4-[6-(aminosulfonyl)-7-chloro-1,2,3,4-tetrahydro-4-oxo-2-quinazolinylphenoxy]-1-(3-mercapto-2-methyl-1-oxopropyl)-L-proline; calcium N[(S)3-(N-cyclohexane-carbonyl-D-alanylthio)-2-methylpropanoyl]-L-prolinate; (−)-(S)1-[(S)3-mercapto-2-methyl-1-oxopropyl]indoline-2-carboxylic acid; and 3-[(5-amino-1-carboxy-1 Spentyl)-amino]-2,3,4,5-tetrahydro-2-oxo-3S 1H1-benzazepine-1-acetic acid.
[58.]The present invention provides the composition of any one of embodiments [1]-[57], further comprising selenium, manganese, glutathione (GSH), L-cysteine, pyridoxine (water soluble Vitamin B₆), riboflavin (water-soluble Vitamin B₂), bioflavenoid (Vitamin P), or any combination thereof.
[59.]The present invention provides a method of treating macular degeneration in a human, the method comprising administering to a human in need of such treatment, an effective amount of the composition of any one of embodiments [1]-[58].
[60.]The present invention provides method of embodiment [58], wherein the macular degeneration is age-related macular degeneration (AMD), early onset macular degeneration, atrophic macular degeneration or neovascular macular degeneration.
[61.]The present invention provides a method of inhibiting angiogenesis in a human, the method comprising administering to a human in need of such inhibition, an effective amount of the composition of any one of embodiments [1]-[58].
[62.]The present invention provides the method of embodiment [61], wherein the angiogenesis causes an angiogenesis dependent disease.
[63.]The present invention provides the method of embodiment [62], wherein the angiogenesis dependent disease is ocular angiogenic diseases, diabetic retinopathy, retinopathy of prematurity, corneal graft rejection, neovascular glaucoma, retrolental fibroplasias, rubeosis, solid tumors, blood born tumors, leukemias, tumor metastases, benign tumors, acoustic neuromas, neurofibromas, trachomas, pyogenic granulomas, rheumatoid arthritis, psoriasis, Osler-Webber Syndrome, myocardial angiogenesis, plaque neovascularization, telangiectasia, hemophiliac joints, angiofibroma, or wound granulation.
[64.]The present invention provides a method for preventing impairment of the vision or for improving impaired vision of a human whose eye has drusen, the method comprising administering to the human in need of such inhibition, an effective amount of the composition of any one of embodiments [1]-[58].
[65.]The present invention provides a method for treating a disease associated with ocular neovascularitis in a human, the method comprising administering to a human in need of such treatment, an effective amount of the composition of any one of embodiments [1]-[58].
[66.]The present invention provides the method of embodiment [65], wherein the disease associated with ocular neovascularitis is epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical bums, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, and corneal graph rejection.
[67.]The present invention provides the method of embodiment [65], wherein the disease associated with ocular neovascularitis is diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications.
[68.]The present invention provides a method of treating cancer in a human, the method comprising administering to a human in need of such treatment, an effective amount of the composition of any one of embodiments [1]-[58].
[69.]The present invention provides the method of embodiment [68], wherein the cancer is colon cancer, prostate cancer, breast cancer, cervical cancer, or skin cancer.
[70.]The present invention provides a method of treating heart disease in a human, the method comprising administering to a human in need of such treatment, an effective amount of the composition of any one of embodiment [1]-[58].
[71.]The present invention provides a method of treating diabetes in a human, the method comprising administering to a human in need of such treatment, an effective amount of the composition of any one of embodiment [1]-[58].
[72.]The present invention provides a method of treating vitamin deficiency in a human, the method comprising administering to a human in need of such treatment, an effective amount of the composition of any one of embodiment [1]-[58].
[73.]The present invention provides the method of any one of embodiments [59]-[72], wherein the administration of the composition is systemic.
[74.]The present invention provides the method of any one of embodiments [59]-[72], wherein the administration of the composition is local.
[75.]The present invention provides the method of any one of embodiments [59]-[72], wherein the administration of the composition is in the form of eye drops, orally, liposomes, parenteral injection, transdermally, aerosolization, inhalant, or direct injection.
[76.]The present invention provides the method of any one of embodiments [59]-[72], wherein the administration of the composition is once daily (QD).
[77.]The present invention provides the method of any one of embodiments [59]-[72], wherein the administration of the composition is at least twice-a-day (BID).
[78.]The present invention provides the method of any one of embodiments [59]-[72], wherein the administration of the composition is carried out for at least one day.
[79.]The present invention provides the method of any one of embodiments [59]-[72], wherein the administration of the composition is carried out for at least one week.
[80.]The present invention provides the method of any one of embodiments [59]-[72], wherein the administration of the composition is carried out for at least one month.
[81.]The present invention provides the method of any one of embodiments [59]-[72], wherein the administration of the composition is carried out for up to about 6 months.
[82.]The present invention provides the method of any one of embodiments [59]-[72], wherein the administration of the composition is carried out for at least about 6 months.

### EXAMPLES

### Example 1

### Chewable Tablet

Each Tablet Contains:

| | | |
|---|---|---|
| Vitamin A | 3500 | IU (29% as Beta Carotene) |
| Vitamin C | 60 | mg |
| Vitamin D | 400 | IU |
| Vitamin E | 30 | IU |
| Vitamin K | 10 | mcg |
| Thiamin | 1.5 | mg |
| Riboflavin | 1.7 | mg |
| Niacin | 20 | mg |
| Vitamin B6 | 2 | mg |
| Folic Acid | 400 | mcg |
| Vitamin B | 12 6 | mcg |
| Biotin | 45 | mcg |
| Pantothenic Acid | 10 | mg |
| Calcium | 108 | mg |
| Iron | 18 | mg |
| Phosphorus | 50 | mg |
| Iodine | 150 | mcg |
| Magnesium | 40 | mg |
| Zinc | 15 | mg |
| Copper | 2 | mg |
| Manganese | 1 | mg |
| Chromium | 20 | mcg |
| Molybdenum | 20 | mcg |
| Free Lutein | 250 | mcg |
| Zeaxanthin | 250 | mcg |

INGREDIENTS: Sucrose, Dibasic Calcium Phosphate, Mannitol, Calcium Carbonate, Stearic Acid, Magnesium Oxide, Ascorbic Acid (Vit. C), Pregelatinized Starch, Microcrystalline Cellulose, dl-Alpha Tocopheryl Acetate (Vit. E). Contains <2% of: Acacia, Aspartame, Beta Carotene, Biotin, BHT, Calcium Pantothenate, Carbonyl Iron, Carrageenan, Chromic Chloride, Citric Acid, Cupric Oxide, Cyanocobalamin (Vit. B12), Dextrose, Ergocalciferol (Vit. D), FD&C Yellow #6 Aluminum Lake, Folic Acid, Gelatin, Glucose, Guar Gum, Lactose, Lutein (free), Magnesium Stearate, Malic Acid, Manganese Sulfate, Mono- and Di-glycerides, Natural and Artificial Flavors, Niacinamide, Phytonadione (Vit. K), Potassium Iodide, Potassium Sorbate, Purified Water, Pyridoxine Hydrochloride (Vit. B6), Riboflavin (Vit. B2), Silicon Dioxide, Sodium Ascorbate, Sodium Benzoate, Sodium Citrate, Sodium Molybdate, Sodium Silicoaluminate, Sorbic Acid, Starch, Thiamine Mononitrate (Vit. B1), Tocopherol, Tribasic Calcium Phosphate, Vanillin, Vitamin A Acetate (Vit. A), Zeaxanthin, Zinc Oxide. May also contain: Fructose, Maltodextrin.

### Example 2

### Chewable Tablet

Each Tablet Contains:

| | | |
|---|---|---|
| Vitamin A | 3500 | IU (29% as Beta Carotene) |
| Vitamin C | 60 | mg |
| Vitamin D | 400 | IU |
| Vitamin E | 45 | IU |
| Vitamin K | 10 | mcg |
| Thiamin | 1.5 | mg |
| Riboflavin | 1.7 | mg |
| Niacin | 20 | mg |
| Vitamin | B6 3 | mg |
| Folic Acid | 400 | mcg |
| Vitamin B12 | 25 | mcg |
| Biotin | 30 | mcg |
| Pantothenic Acid | 10 | mg |
| Calcium | 200 | mg |
| Phosphorus | 48 | mg |
| Iodine | 150 | mcg |
| Magnesium | 100 | mg |
| Zinc | 15 | mg |
| Selenium | 20 | mcg |
| Copper | 2 | mg |
| Manganese | 2 | mg |
| Chromium | 150 | mcg |
| Molybdenum | 75 | mcg |
| Chloride | 72 | mg |
| Potassium | 80 | mg |
| Boron | 150 | mcg |
| Nickel | 5 | mcg |
| Silicon | 2 | mg |
| Vanadium | 10 | mcg |
| Lutein (free) | 250 | mcg |
| Zeaxanthin | 250 | mcg |
| Lycopene | 300 | mcg |

INGREDIENTS: Calcium Carbonate, Dibasic Calcium Phosphate, Magnesium Oxide, Potassium Chloride, Microcrystalline Cellulose, Ascorbic Acid (Vit. C), dl-Alpha Tocopheryl Acetate (Vit. E), Gelatin, Pregelatinized Starch, Crospovidone. Contains less than 2% of the following: Acacia Senegal Gum, Ascorbyl Palmitate, Beta Carotene, Biotin, Boron, Butylated Hydroxytoluene, Calcium Pantothenate, Chromic Chloride, Citric Acid, Colloidal Silicon Dioxide, Cupric Oxide, Cyanocobalamin (Vit. B12), Ergocalciferol (Vit. D), FD&C Blue No. 2 Aluminum Lake, FD&C Red No. 40 Aluminum Lake, FD&C Yellow No. 6 Aluminum Lake, Folic Acid, Hypromellose, Lutein (free), Lycopene, Magnesium Stearate, Manganese Sulfate, Niacinamide, Nickelous Sulfate, Phytonadione (Vit. K), Polysorbate 80, Potassium Iodide, Potassium Sorbate, Purified Water, Pyridoxine Hydrochloride (Vit. B6), Riboflavin (Vit. B2), Silicon Dioxide, Sodium Ascorbate, Sodium Benzoate, Sodium Citrate, Sodium Metavanadate, Sodium Molybdate, Sodium Selenate, Sodium Silicoaluminate, Sorbic Acid, Starch, Sucrose, Thiamine Mononitrate (Vit. B1), Titanium Dioxide, Tocopherol, Tribasic Calcium Phosphate, Triethyl Citrate, Vitamin A Acetate (Vit. A), Zeaxanthin, Zinc Oxide. May also contain: Calcium Stearate, Glucose, Lactose Monohydrate.

### Example 3

### Chewable Tablet

Each Tablet Contains:

| | | |
|---|---|---|
| Vitamin A | 3500 | IU (29% as Beta Carotene) |
| Vitamin C | 120 | mg |
| Vitamin D | 400 | IU |
| Vitamin E | 60 | IU |
| Vitamin K | 25 | mcg |
| Thiamin | 4.5 | mg |
| Riboflavin | 5.1 | mg |
| Niacin | 40 | mg |
| Vitamin B6 | 6 | mg |
| Folic Acid | 400 | mcg |
| Vitamin B12 | 18 | mcg |
| Biotin | 40 | mcg |
| Pantothenic Acid | 10 | mg |
| Calcium | 100 | mg |
| Iron | 18 | mg |
| Phosphorus | 48 | mg |
| Iodine | 150 | mcg |
| Magnesium | 40 | mg |
| Zinc | 15 | mg |
| Selenium | 70 | mcg |
| Copper | 2 | mg |
| Manganese | 4 | mg |
| Chromium | 120 | mcg |
| Molybdenum | 75 | mcg |
| Chloride | 72 | mg |
| Potassium | 80 | mg |
| Ginseng Root | 50 | mg Standardized Extract |
| (Panax ginseng) | | |
| Ginkgo Biloba Leaf | 60 | mg Standardized Extract |
| (Ginkgo biloba) | | |
| Boron | 60 | mcg |
| Nickel | 5 | mcg |
| Silicon | 4 | mg |
| Tin | 10 | mcg |
| Vanadium | 10 | mcg |
| Lutein (free) | 250 | mcg |
| Zeaxanthin | 250 | mcg |

INGREDIENTS: Dibasic Calcium Phosphate, Potassium Chloride, Ascorbic Acid (Vit. C), Microcrystalline Cellulose, Calcium Carbonate, dl-Alpha Tocopheryl Acetate (Vit. E), Magnesium Oxide, Ginkgo Biloba Leaf (Ginkgo biloba) Standardized Extract, Gelatin, Ginseng Root (Panax ginseng) Standardized Extract, Ferrous Fumarate, Niacinamide, Crospovidone, Starch. Contains <2% of: Acacia Senegal Gum, Beta Carotene, Biotin, BHT, Calcium Pantothenate, Chromic Chloride, Citric Acid, Cupric Oxide, Cyanocobalamin (Vit. B12), Ergocalciferol (Vit. D), FD&C Red #40 Aluminum Lake, FD&C Yellow #6 Aluminum Lake, Folic Acid, Glucose, Hypromellose, Lactose Monohydrate, Lutein (free), Magnesium Borate, Magnesium Stearate, Manganese Sulfate, Nickelous Sulfate, Phytonadione (Vit. K), Polyethylene Glycol, Polysorbate 80, Potassium Iodide, Potassium Sorbate, Purified Water, Pyridoxine Hydrochloride (Vit. B6), Riboflavin (Vit. B2), Silicon Dioxide, Sodium Ascorbate, Sodium Benzoate, Sodium Borate, Sodium Citrate, Sodium Metavanadate, Sodium Molybdate, Sodium Selenate, Sodium Silicoaluminate, Sorbic Acid, Stannous Chloride, Sucrose, Thiamin Mononitrate (Vit. BI), Titanium Dioxide, Tocopherol, Tribasic Calcium Phosphate, Vitamin A Acetate (Vit. A), Zeaxanthin, Zinc Oxide. May also contain: Maltodextrin.

### Example 4

### Chewable Tablet

Each Tablet Contains:

| | | |
|---|---|---|
| Vitamin A | 3500 | IU (29% as Beta Carotene) |
| Vitamin C | 60 | mg |
| Vitamin D | 400 | IU |
| Vitamin E | 30 | IU |
| Vitamin K | 10 | mcg |
| Thiamin | 1.5 | mg |
| Riboflavin | 1.7 | mg |
| Niacin | 20 | mg |
| Vitamin B6 | 2 | mg |
| Folic Acid | 400 | mcg |
| Vitamin B | 12 6 | mcg |
| Biotin | 45 | mcg |
| Pantothenic Acid | 10 | mg |
| Calcium | 108 | mg |
| Iron | 18 | mg |
| Phosphorus | 50 | mg |
| Iodine | 150 | mcg |
| Magnesium | 40 | mg |
| Zinc | 15 | mg |
| Copper | 2 | mg |
| Manganese | 1 | mg |
| Chromium | 20 | mcg |
| Molybdenum | 20 | mcg |
| Free Lutein | 500 | mcg |
| Zeaxanthin | 500 | mcg |

INGREDIENTS: Sucrose, Dibasic Calcium Phosphate, Mannitol, Calcium Carbonate, Stearic Acid, Magnesium Oxide, Ascorbic Acid (Vit. C), Pregelatinized Starch, Microcrystalline Cellulose, dl-Alpha Tocopheryl Acetate (Vit. E). Contains <2% of: Acacia, Aspartame, Beta Carotene, Biotin, BHT, Calcium Pantothenate, Carbonyl Iron, Carrageenan, Chromic Chloride, Citric Acid, Cupric Oxide, Cyanocobalamin (Vit. B12), Dextrose, Ergocalciferol (Vit. D), FD&C Yellow #6 Aluminum Lake, Folic Acid, Gelatin, Glucose, Guar Gum, Lactose, Lutein (free), Magnesium Stearate, Malic Acid, Manganese Sulfate, Mono- and Di-glycerides, Natural and Artificial Flavors, Niacinamide, Phytonadione (Vit. K), Potassium Iodide, Potassium Sorbate, Purified Water, Pyridoxine Hydrochloride (Vit. B6), Riboflavin (Vit. B2), Silicon Dioxide, Sodium Ascorbate, Sodium Benzoate, Sodium Citrate, Sodium Molybdate, Sodium Silicoaluminate, Sorbic Acid, Starch, Thiamine Mononitrate (Vit. B1), Tocopherol, Tribasic Calcium Phosphate, Vanillin, Vitamin A Acetate (Vit. A), Zeaxanthin, Zinc Oxide. May also contain: Fructose, Maltodextrin.

### Example 5

### Chewable Tablet

Each Tablet Contains:

| | | |
|---|---|---|
| Vitamin A | 3500 | IU (29% as Beta Carotene) |
| Vitamin C | 60 | mg |
| Vitamin D | 400 | IU |
| Vitamin E | 45 | IU |
| Vitamin K | 10 | mcg |
| Thiamin | 1.5 | mg |
| Riboflavin | 1.7 | mg |
| Niacin | 20 | mg |
| Vitamin | B6 3 | mg |
| Folic Acid | 400 | mcg |
| Vitamin B12 | 25 | mcg |
| Biotin | 30 | mcg |
| Pantothenic Acid | 10 | mg |
| Calcium | 200 | mg |
| Phosphorus | 48 | mg |
| Iodine | 150 | mcg |
| Magnesium | 100 | mg |
| Zinc | 15 | mg |
| Selenium | 20 | mcg |
| Copper | 2 | mg |
| Manganese | 2 | mg |
| Chromium | 150 | mcg |
| Molybdenum | 75 | mcg |
| Chloride | 72 | mg |
| Potassium | 80 | mg |
| Boron | 150 | mcg |
| Nickel | 5 | mcg |
| Silicon | 2 | mg |
| Vanadium | 10 | mcg |
| Lutein (free) | 500 | mcg |
| Zeaxanthin | 500 | mcg |
| Lycopene | 300 | mcg |

INGREDIENTS: Calcium Carbonate, Dibasic Calcium Phosphate, Magnesium Oxide, Potassium Chloride, Microcrystalline Cellulose, Ascorbic Acid (Vit. C), dl-Alpha Tocopheryl Acetate (Vit. E), Gelatin, Pregelatinized Starch, Crospovidone. Contains less than 2% of the following: Acacia Senegal Gum, Ascorbyl Palmitate, Beta Carotene, Biotin, Boron, Butylated Hydroxytoluene, Calcium Pantothenate, Chromic Chloride, Citric Acid, Colloidal Silicon Dioxide, Cupric Oxide, Cyanocobalamin (Vit. B12), Ergocalciferol (Vit. D), FD&C Blue No. 2 Aluminum Lake, FD&C Red No. 40 Aluminum Lake, FD&C Yellow No. 6 Aluminum Lake, Folic Acid, Hypromellose, Lutein (free), Lycopene, Magnesium Stearate, Manganese Sulfate, Niacinamide, Nickelous Sulfate, Phytonadione (Vit. K), Polysorbate 80, Potassium Iodide, Potassium Sorbate, Purified Water, Pyridoxine Hydrochloride (Vit. B6), Riboflavin (Vit. B2), Silicon Dioxide, Sodium Ascorbate, Sodium Benzoate, Sodium Citrate, Sodium Metavanadate, Sodium Molybdate, Sodium Selenate, Sodium Silicoaluminate, Sorbic Acid, Starch, Sucrose, Thiamine Mononitrate (Vit. B1), Titanium Dioxide, Tocopherol, Tribasic Calcium Phosphate, Triethyl Citrate, Vitamin A Acetate (Vit. A), Zeaxanthin, Zinc Oxide. May also contain: Calcium Stearate, Glucose, Lactose Monohydrate.

### Example 6

### Chewable Tablet

Each Tablet Contains:

| | | |
|---|---|---|
| Vitamin A | 3500 | IU (29% as Beta Carotene) |
| Vitamin C | 120 | mg |
| Vitamin D | 400 | IU |
| Vitamin E | 60 | IU |
| Vitamin K | 25 | mcg |
| Thiamin | 4.5 | mg |
| Riboflavin | 5.1 | mg |
| Niacin | 40 | mg |
| Vitamin B6 | 6 | mg |
| Folic Acid | 400 | mcg |
| Vitamin B12 | 18 | mcg |
| Biotin | 40 | mcg |
| Pantothenic Acid | 10 | mg |
| Calcium | 100 | mg |
| Iron | 18 | mg |
| Phosphorus | 48 | mg |
| Iodine | 150 | mcg |
| Magnesium | 40 | mg |
| Zinc | 15 | mg |
| Selenium | 70 | mcg |
| Copper | 2 | mg |
| Manganese | 4 | mg |
| Chromium | 120 | mcg |
| Molybdenum | 75 | mcg |
| Chloride | 72 | mg |
| Potassium | 80 | mg |
| Ginseng Root | 50 | mg Standardized Extract |
| (Panax ginseng) | | |
| Ginkgo Biloba Leaf | 60 | mg Standardized Extract |
| (Ginkgo biloba) | | |
| Boron | 60 | mcg |
| Nickel | 5 | mcg |
| Silicon | 4 | mg |
| Tin | 10 | mcg |
| Vanadium | 10 | mcg |
| Lutein (free) | 500 | mcg |
| Zeaxanthin | 500 | mcg |

INGREDIENTS: Dibasic Calcium Phosphate, Potassium Chloride, Ascorbic Acid (Vit. C), Microcrystalline Cellulose, Calcium Carbonate, dl-Alpha Tocopheryl Acetate (Vit. E), Magnesium Oxide, Ginkgo Biloba Leaf (Ginkgo biloba) Standardized Extract, Gelatin, Ginseng Root (Panax ginseng) Standardized Extract, Ferrous Fumarate, Niacinamide, Crospovidone, Starch. Contains <2% of: Acacia Senegal Gum, Beta Carotene, Biotin, BHT, Calcium Pantothenate, Chromic Chloride, Citric Acid, Cupric Oxide, Cyanocobalamin (Vit. B12), Ergocalciferol (Vit. D), FD&C Red #40 Aluminum Lake, FD&C Yellow #6 Aluminum Lake, Folic Acid, Glucose, Hypromellose, Lactose Monohydrate, Lutein (free), Magnesium Borate, Magnesium Stearate, Manganese Sulfate, Nickelous Sulfate, Phytonadione (Vit. K), Polyethylene Glycol, Polysorbate 80, Potassium Iodide, Potassium Sorbate, Purified Water, Pyridoxine Hydrochloride (Vit. B6), Riboflavin (Vit. B2), Silicon Dioxide, Sodium Ascorbate, Sodium Benzoate, Sodium Borate, Sodium Citrate, Sodium Metavanadate, Sodium Molybdate, Sodium Selenate, Sodium Silicoaluminate, Sorbic Acid, Stannous Chloride, Sucrose, Thiamin Mononitrate (Vit. B1), Titanium Dioxide, Tocopherol, Tribasic Calcium Phosphate, Vitamin A Acetate (Vit. A), Zeaxanthin, Zinc Oxide. May also contain: Maltodextrin.

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

It is appreciated that certain features of the invention, which are, for clarity, described in the context of separate embodiments, may also be provided in combination in a single embodiment. Conversely, various features of the invention which are for brevity, described in the context of a single embodiment, may also be provided separately or in any sub-combination.

1. A composition comprising:
(a) non-esterified lutein;
(b) zeaxanthin;
(c) vitamin C;
(d) vitamin E;
(e) zinc; and
(f) copper.

2. The composition of claim 1, wherein the vitamin C is provided in the form of ascorbic acid.

3. The composition of claim 1, wherein the vitamin E is provided in the form of dl-alpha tocopheryl acetate.

4. The composition of claim 1, wherein the zinc is provided in the form of zinc oxide, zinc gluconate or a combination thereof.

5. The composition of claim 1, wherein the copper is provided in the form of cupric oxide, copper gluconate or a combination thereof.

6. The composition of claim 1, wherein the composition is provided as a daily dosage.

7. The composition of claim 1, wherein the vitamin C is present in about 200 mg to about 800 mg.

8. The composition of claim 1, wherein the vitamin E is present in about 200 IU to about 700 IU.

9. The composition of claim 1, wherein the zinc is present in about 40 mg to about 150 mg.

10. The composition of claim 1, wherein the copper is present in about 1.6 mg to about 2.4 mg.

11. The composition of claim 1, further comprising an anti-inflammatory steroid.

12. The composition of claim 1, further comprising a non-steroidal anti-inflammatory drug (NSAID).

13. The composition of claim 1, further comprising selenium, manganese, glutathione (GSH), L-cysteine, pyridoxine (water soluble Vitamin B₆), riboflavin (water-soluble Vitamin B₂), bioflavenoid (Vitamin P), or any combination thereof.

14. A method of treating macular degeneration in a human, the method comprising administering to a human in need of such treatment, an effective amount of the composition of claim 1.

15. The method of claim 14, wherein the macular degeneration is age-related macular degeneration (AMD), early onset macular degeneration, atrophic macular degeneration or neovascular macular degeneration.

16. A method of inhibiting angiogenesis in a human, the method comprising administering to a human in need of such inhibition, an effective amount of the composition of claim 1.

17. The method of claim 16, wherein the angiogenesis causes an angiogenesis dependent disease.

18. The method of claim 17, wherein the angiogenesis dependent disease is ocular angiogenic diseases, diabetic retinopathy, retinopathy of prematurity, corneal graft rejection, neovascular glaucoma, retrolental fibroplasias, rubeosis, solid tumors, blood born tumors, leukemias, tumor metastases, benign tumors, acoustic neuromas, neurofibromas, trachomas, pyogenic granulomas, rheumatoid arthritis, psoriasis, Osler-Webber Syndrome, myocardial angiogenesis, plaque neovascularization, telangiectasia, hemophiliac joints, angiofibroma, or wound granulation.

19. A method for preventing impairment of the vision or for improving impaired vision of a human whose eye has drusen, the method comprising administering to the human in need of such inhibition, an effective amount of the composition of claim 1.

20. A method for treating a disease associated with ocular neovascularitis in a human, the method comprising administering to a human in need of such treatment, an effective amount of the composition claim 1.

21. The method of claim 20, wherein the disease associated with ocular neovascularitis is epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical bums, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, and corneal graph rejection.

22. The method of claim 21, wherein the disease associated with ocular neovascularitis is diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications.

23. A method of treating cancer in a human, the method comprising administering to a human in need of such treatment, an effective amount of the composition of claim 1.

24. The method of claim 23, wherein the cancer is colon cancer, prostate cancer, breast cancer, cervical cancer, or skin cancer.

25. A method of treating heart disease in a human, the method comprising administering to a human in need of such treatment, an effective amount of the composition of claim 1.

26. A method of treating diabetes in a human, the method comprising administering to a human in need of such treatment, an effective amount of the composition of claim 1.

27. A method of treating vitamin deficiency in a human, the method comprising administering to a human in need of such treatment, an effective amount of the composition of claim 1.
